(19) United States
(12) Patent Application Publication
(10) Pub. No.: US 2002/0051819 A1
(43) Pub. Date: May 2, 2002

(54) Peptides, compositions and methods for the treatment of burkholderia cepacia

(76) Inventors: Carla H. Kuhner, Avondale, PA (US); James A. Romesser, Kennett Square, PA (US)

Correspondence Address: WOODCOCK WASHBURN KURTZ, MACKIEWICZ & NORRIS LLP, 46th Floor, One Liberty Place, Philadelphia, PA, 19103, US

(21) Appl. No.: 09/881,954

(22) Filed: Jun. 15, 2001

Related U.S. Application Data
(60) Provisional application No. 60/212,440, filed on Jun. 16, 2000.

(51) Int. Cl. A61K 9/14; A61K 38/08

(52) U.S. Cl. 424/484; 514/17; 424/486; 424/488

(57) ABSTRACT

Peptides, compositions and methods for inhibiting and controlling the growth of Burkholderia cepacia are disclosed. The composition comprises a peptide mixture with antimicrobial activity against Burkholderia cepacia and at least one carrier. The method comprises delivering an amount, effective for the prevention, inhibition and termination of the growth of Burkholderia cepacia for industrial, pharmaceutical, household, and personal care use.

### BACKGROUND OF THE INVENTION

1. Field of the Invention P This invention relates to peptides possessing antimicrobial activity and methods of using them to combat microbes. Peptides of the present invention are particularly useful in the treatment of Burkholderia cepacia in industrial and clinical environments.

2. Background of the Invention and Related Information

Peptides are now recognized as part of a global defense mechanism used by animals and plants in terrestrial and marine environments to prevent microbial attack. The discovery of antimicrobial peptides has generated interest in the use of these compounds to combat clinically relevant microorganisms, in particular, multi-drug resistant organisms. Large screening programs have been developed to identify potential peptide-based drug candidates from both natural product-and combinatorial chemistry-derived libraries. Antimicrobial peptides are also potential candidates for the prevention of biofouling in industrial water systems, where they would represent a novel chemical class of antibiofouling compounds.

Peptides are produced naturally in bacteria, fungi, plants, insects, amphibians, crustaceans, fish and mammals [Hancock, Advances in Microbial Physiology, 135-175, Academic Press (1995)]. They represent a major inducible defense against microbes and their production in the immune system of many species is controlled by transcriptional elements. For instance, in humans, antimicrobial peptides are found in neutrophils which are responsible for responding against invasion of foreign organisms [Lehrer et al. ASM News, 56, 315-318, (1990)]. Natural antimicrobial peptides have a moderate spectrum of activity against microbes and are usually present in moderate amounts. Natural antimicrobial peptides of 12-50 amino acid residues have been obtained in the past 20 years via isolation from the defense systems of insects, amphibians and mammals [Oh et al. J. Peptide Res., 56, 41-46, (1998)]. Use of these peptides in clinical trials has shown effective antimicrobial activity [Hancock, Exp. Opin. Invest. Drugs, 7, 167-174, (1998)].

Treatment of microorganisms with antibiotics has resulted in inadequate inhibition of bacterial growth due to resistance. Peptides have shown excellent activity against antibiotic resistant microorganisms in vitro [Hancock and Lehrer, TiB Tech., 16, 82-88, (1998)].

The charge distribution and hydrophobic properties of a peptide appear to be important factors in determining its effectiveness. The peptides are usually large (12-50 amino acids) and said to be cationic due to the presence of positively charged basic amino acid residues such as arginine and lysine [Hancock, Exp. Opin. Invest. Drugs, 7, 167-174, (1998)]. It is suggested that the cationicity of the peptide may play an important role in the peptide interaction with negatively charged membranes. For instance, cationic peptides are said to compete with divalent cations on the surface of Gram-negative bacteria and prevent their interaction with lipopolysaccharide (LPS) molecules [Hancock, Exp. Opin. Invest. Drugs, 7, 167-174, (1998)]. It is hypothesized that the displacement of divalent cations by cationic peptides creates a distortion in the outer membrane of the bacteria through which peptides may pass.

Industrial facilities employ many methods of preventing biofouling of industrial water systems. Many microbial organisms are involved in biofilm formation in industrial waters. Growth of slime-producing bacteria in industrial water systems causes problems including decreased heat transfer, fouling and blockage of lines and valves, and corrosion or degradation of surfaces. Control of bacterial growth in the past has been accomplished with biocides. Many biocides and biocide formulations are known in the art. However, many of these contain components which may be environmentally deleterious or toxic, and are often resistant to breakdown.

The manufacturing cost of peptides may be a limiting factor in their antimicrobial application [Hancock and Lehrer, TiB Tech., 16, 82-88, (1998)]. The long chain length of the natural antimicrobial peptides is a major factor contributing to their cost of synthesis.

U.S. Pat. No. 5,504,190 describes a process for solid-support synthesis of equimolar oligomer mixtures that prevents unequal reaction yields during addition of blocked amino acids and allows for equal and precise representation of amino acid residues along the chain of the peptide. The peptides synthesized are said to exhibit antimicrobial activity, and contain equimolar amounts of preferably at least 6 amino acid residues. The peptides disclosed include 6-mer oligopeptide mixtures beginning with Ac-Arg-Arg-, Ac-Trp-Trp-, Ac-Cys-Cys-, Ac-Trp-Cys-, Ac-Trp-Leu-, Ac-Trp-Lys-, Ac-Arg-Trp-, Ac-Thr-Arg-, Ac-Gln-Tyr-, and Ac-Arg-Met-.

U.S. Pat. No. 5,786,324 discloses peptides that are minimally 10 amino acids long and are lysine and arginine rich. These peptides showed antimicrobial activity against Gram-negative bacteria including Pseudomonas aeruginosa but were not active against Burkholderia cepacia.

U.S. Pat. No. 5,736,533 discloses an oligosaccharide compound which is said to be effective against bacteria consisting of Streptococcus pneumoniae, Haemophilus influenza, Haemophilus parainfluenza, and Burkholderia cepacia.

Industrial plants have been concerned with methods to prevent biofouling of industrial water systems. Many microbial organisms, including Burkholderia cepacia, are involved in the biofilm formation in industrial water systems. Growth of slime-producing bacteria in industrial water systems causes problems including decreased heat transfer, fouling and blockage of lines and valves, and corrosion or degradation of surfaces.

Burkholderia is also a nosocomial pathogen and causes infections due to contaminated equipment, medications and disinfectants. Infections include bacteremia due to contamination of indwelling catheters, urinary tract infection, peritonitis and respiratory tract infection.

B. cepacia is an important pathogen in cystic fibrosis and chronic granulomatous disease. In cystic fibrosis patients, B. cepacia is the major organism responsible for morbidity and mortality.

B. cepacia is one of the most antibiotic resistant organisms isolated in the clinical laboratory. The present invention provides safe and effective peptides with activity against Burkholderia cepacia for use in clinical and industrial settings.

### SUMMARY OF THE INVENTION

The invention provides antimicrobial compositions comprising a plurality of peptides, wherein said peptides each are represented by Formula I:

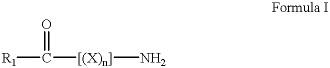

wherein:

X represents any amino acid except glutamate or aspartate;

n=1-10;

R₁ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₁-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;

R₃ is independently hydrogen; C₁-C₄ alkyl; or phenyl optionally substituted with at least one R₈;

R₄ is independently hydrogen; C₁-C₈ alkyl; or phenyl optionally substituted with at least one R₈;

R₅ is independently C₁-C₆ alkyl; C₁-C₆ alkoxy; C₁-C₆ haloalkyl; halogen; C₂-C₈ alkynyl; C₁-C₆ thioalkyl; phenyl or phenoxy each optionally substituted with at least one R₈; cyano; nitro; C₁-C₆ haloalkoxy; C₁-C₆ haloalkythio; C₂-C₆ alkenyl; C₂-C₆ haloalkenyl; acetyl; CO₂CH₃; or N(C₁-C₂ alkyl)₂;

R₆ is independently methyl; ethyl; methoxy; methylthio; halogen; or trifluoromethyl;

R₇ is independently halogen; and

R₈ is independently halogen; C₁-C₄ alkyl; C₁-C₄ alkoxy; C₁-C₄ haloalkyl; nitro; or cyano;

wherein:

the amino acid in the first position, based on numbered amino acids from N-terminus to C-terminus, is selected from the group consisting of arginine, lysine, methionine, serine, threonine and tryptophan;

the amino acid in the second position, based on numbered amino acids from N-terminus to C-terminus, is selected from the group consisting of arginine, histidine, cysteine, threonine, tyrosine, and tryptophan; and

the amino acids in positions three through six, based on numbered amino acids from N-terminus to C-terminus, are any amino acid;

wherein the first two amino acids of said hexapeptides are other than arginine arginine, tryptophan-tryptophan, tryptophan-cysteine, tryptophan-lysine, arginine-tryptophan, or threonine-arginine.

The invention also provides antimicrobial compositions comprising a plurality of peptides, wherein said peptides each are represented by Formula II:

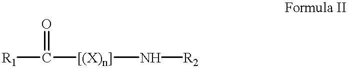

wherein:

X represents any amino acid except glutamate or aspartate;

n=1-10;

R₁ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₅-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;

R₂ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₅-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;

R₃ is independently hydrogen; C₁-C₄ alkyl; or phenyl optionally substituted with at least one R₈;

R₄ is independently hydrogen; C₁-C₈ alkyl; or phenyl optionally substituted with at least one R₈;

R₅ is independently C₁-C₆ alkyl; C₁-C₆ alkoxy; C₁-C₆ haloalkyl; halogen; C₂-C₈ alkynyl; C₁-C₆ thioalkyl; phenyl or phenoxy each optionally substituted with at least one R₈; cyano; nitro; C₁-C₆ haloalkoxy; C₁-C₆ haloalkythio; C₂-C₆ alkenyl; C₂-C₆ haloalkenyl; acetyl; CO₂CH₃; or N(C₁-C₂ alkyl)₂;

R₆ is independently methyl; ethyl; methoxy; methylthio; halogen; or trifluoromethyl;

R₇ is independently halogen; and

R₈ is independently halogen; C₁-C₄ alkyl; C₁-C₄ alkoxy; C₁-C₄ haloalkyl; nitro; or cyano;

wherein:

the amino acid in the first position, based on numbered amino acids from N-terminus to C-terminus, is selected from the group consisting of arginine, lysine, methionine, serine, threonine and tryptophan;

the amino acid in the second position, based on numbered amino acids from N-terminus to C-terminus, is selected from the group consisting of arginine, histidine, cysteine, threonine, tyrosine, and tryptophan; and

the amino acids in positions three through six, based on numbered amino acids from N-terminus to C-terminus, are any amino acid.

In some embodiments, the invention provides antimicrobial compositions comprising a plurality of hexapeptides, wherein for each hexapeptide, the amino acid in the first position, based on numbered amino acids from N-terminus to C-terminus, is selected from the group consisting of arginine, lysine, methionine, serine, threonine and tryptophan; the amino acid in the second position, based on numbered amino acids from N-terminus to C-terminus, is selected from the group consisting of arginine, histidine, cysteine, threonine, tyrosine, and tryptophan; the amino acids in positions three through six, based on numbered amino acids from N-terminus to C-terminus, are any amino acid; and wherein the first two amino acids of said hexapeptides are other than arginine-arginine, tryptophan-tryptophan, tryptophan-cysteine, tryptophan-lysine, arginine-tryptophan, or threonine-arginine.

In some embodiments, the invention provides antimicrobial compositions comprising a plurality of peptides, wherein said peptides each are represented by Formula I:

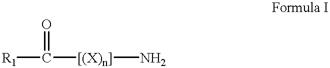

wherein:

X represents any amino acid except glutamate or aspartate;

n=1-10;

R₁ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₅-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;

R₃ is independently hydrogen; C₁-C₄ alkyl; or phenyl optionally substituted with at least one R₈;

R₄ is independently hydrogen; C₁-C₈ alkyl; or phenyl optionally substituted with at least one R₈;

R₅ is independently C₁-C₆ alkyl; C₁-C₆ alkoxy; C₁-C₆ haloalkyl; halogen; C₂-C₈ alkynyl; C₁-C₆ thioalkyl; phenyl or phenoxy each optionally substituted with at least one R₈; cyano; nitro; C₁-C₆ haloalkoxy; C₁-C₆ haloalkythio; C₂-C₆ alkenyl; C₂-C₆ haloalkenyl; acetyl; CO₂CH₃; or N(C₁-C₂ alkyl)₂;

R₆ is independently methyl; ethyl; methoxy; methylthio; halogen; or trifluoromethyl;

R₇ is independently halogen; and

R₈ is independently halogen; C₁-C₄ alkyl; C₁-C₄ alkoxy; C₁-C₄ haloalkyl; nitro; or cyano;

wherein:

the amino acid in the first position, based on numbered amino acids from N-terminus to C-terminus, is selected from the group consisting of arginine, lysine, methionine, serine, threonine and tryptophan;

the amino acid in the second position, based on numbered amino acids from N-terminus to C-terminus, is selected from the group consisting of arginine, histidine, cysteine, threonine, tyrosine, and tryptophan;

the amino acids in positions three through six, based on numbered amino acids from N-terminus to C-terminus, are any amino acid; and

wherein the first two amino acids of said hexapeptides are other than arginine-arginine, tryptophan-tryptophan, tryptophan-cysteine, tryptophan-lysine, arginine-tryptophan, or threonine-arginine.

In other embodiments, the invention provides antimicrobial compositions comprising a plurality of peptides, wherein said peptides each are represented by Formula II:

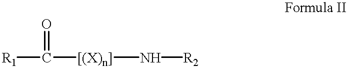

wherein:

X represents any amino acid except glutamate or aspartate;

n=6;

R₁ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₅-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;

R₂ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₁-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;

R₃ is independently hydrogen; C₁-C₄ alkyl; or phenyl optionally substituted with at least one R₈;

R₄ is independently hydrogen; C₁-C₈ alkyl; or phenyl optionally substituted with at least one R₈;

R₅ is independently C₁-C₆ alkyl; C₁-C₆ alkoxy; C₁-C₆ haloalkyl; halogen; C₂-C₈ alkynyl; C₁-C₆ thioalkyl; phenyl or phenoxy each optionally substituted with at least one R₈; cyano; nitro; C₁-C₆ haloalkoxy; C₁-C₆ haloalkythio; C₂-C₆ alkenyl; C₂-C₆ haloalkenyl; acetyl; CO₂CH₃; or N(C₁-C₂ alkyl)₂;

R₆ is independently methyl; ethyl; methoxy; methylthio; halogen; or trifluoromethyl;

R₇ is independently halogen; and

R₈ is independently halogen; C₁-C₄ alkyl; C₁-C₄ alkoxy; C₁-C₄ haloalkyl; nitro; or cyano;

wherein:

the amino acid in the first position, based on numbered amino acids from N-terminus to C-terminus, is selected from the group consisting of arginine, lysine, methionine, serine, threonine and tryptophan;

the amino acid in the second position, based on numbered amino acids from N-terminus to C-terminus, is selected from the group consisting of arginine, histidine, cysteine, threonine, tyrosine, and tryptophan; and

the amino acids in positions three through six, based on numbered amino acids from N-terminus to C-terminus, are any amino acid.

In some embodiments, the compositions comprise hexapeptides wherein the amino acids in the first and second positions of said peptides, based on numbered amino acids from N-terminus to C-terminus, are selected from the group consisting of Arg-Tyr, Arg-Cys, Ser-Thr, Met-Trp, Lys-Trp, Thr-Trp, Trp-Arg, Trp-His, and Trp-Tyr.

The peptides may be incorporated into a polymer, including, but not limited to a polysaccharide, a glycol polymer, a polyester, a polyurethane, a polyacrylate, a polyacrylonitrile, a polyamide, a polyolefin, a polystyrene, a vinyl polymer, a polypropylene, silk, a biopolymer, and mixtures thereof.

The invention also provides compositions comprising the antimicrobial peptides and at least one carrier, including, but not limited to a pharmaceutically acceptable carrier, an industrially acceptable carrier, a household product, and a personal care composition.

In the compositions of the invention, peptides may be present in an amount of about 0.000001 to about 99% based on the weight percentage of the composition. In some embodiments, the peptides are present in an amount of about 0.001 to about 50% based on the weight percentage of the composition. In other embodiments, the peptides are present in an amount of about 0.01 to about 25% based on the weight percentage of the composition.

In the compositions of the invention, the carrier may be present in an amount of, for example, about 1 to about 99% based on the weight percentage of said composition. In some embodiments, the carrier is present in an amount of about 50 to about 99% based on the weight percentage of said composition. In other embodiments, the carrier is present in an amount of about 75 to about 99% based on the weight percentage of said composition.

The invention also provides methods for preventing, inhibiting, or terminating the growth of at least one microbe comprising administering the antimicrobial peptides and compositions of the invention.

The methods are effective against microbes, including, for example, bacteria, archaea, fungi, algae, protozoa, multicellular parasites, and viruses.

In some embodiments, the methods of the invention are useful against Gram-positive cocci, Gram-negative cocci, Gram-positive straight rods, Gram-negative straight rods, Gram-positive curved rods, Gram-negative curved rods, Gram-positive helical/vibroid rods, Gram-negative helical/vibroid rods, Gram-positive branched rods, Gram-negative branched rods, sheathed bacteria, sulfur-oxidizing bacteria, sulfur or sulfate-reducing bacteria, spirochetes, actinomycetes, myxobacteria, mycoplasmas, rickettsias, chlamydias, cyanobacteria, archea, fungi, parasites, viruses and algae.

In other embodiments, the methods of the invention are useful against Burkholderia cepacia.

In some embodiments of the methods of the invention, the antimicrobial compositions are administered enterally. A dosage may be, for example, about 0.01 to about 100 mg/kg.

In other embodiments of the methods of the invention, the antimicrobial compositions are administered parenterally. A dosage may be, for example, about 0.01 to about 100 mg/kg.

In other embodiments of the methods of the invention, the antimicrobial compositions are administered topically. A typical dosage may be, for example, about 0.000001 to about 20% based on the weight of the composition.

In further embodiments of the invention, the antimicrobial compositions are administered to an aqueous environment comprising at least one biofouling microbe. In the administration of the compositions to aqueous environments, the peptides may be present in an amount of, for example, about 0.001 to about 50% based on the weight percentage of the composition.

The antimicrobial compositions of the invention may also be used as coatings for substrates, including, but not limited to personal care products, healthcare products, household products, food preparation surfaces, food packaging surfaces, medical devices, wound dressings, surgical staples, membranes, shunts, surgical gloves, tissue patches, prosthetic devices, wound drainage tubes, blood collection and transfer devices, tracheotomy devices, intraocular lenses, laboratory devices, and textile products. The invention also provides substrates coated with the antimicrobial compositions of the invention.

These, as well as other, aspects of the invention are set forth in greater detail below.

### BRIEF DESCRIPTION OF DRAWINGS

The foregoing and other objects, features and advantages of the invention will be apparent from the following more particular description of the preferred embodiments, as illustrated in the accompanying drawing, and wherein:

FIG. 1 is a table demonstrating growth of Burkholderia cepacia in the presence of hexapeptide mixtures comprising equimolar concentrations of peptides with defined L-amino acids in positions 1 and 2 and undefined (any of the 20 naturally occurring amino acids) in positions 3, 4, 5 and 6. The first column shows the amino acids in the first two positions of each hexapeptide mixture. The second column is the concentration of hexapeptides assayed in parts per million (ppm). The third column is the percent growth inhibition of Burkholderia cepacia by the hexapeptide mixtures at a concentration of 625 ppm.

### DETAILED DESCRIPTION OF THE INVENTION

The present invention is directed to peptides possessing antimicrobial activity. Peptides of the present invention may be used to combat microbes which include, but are not limited to, Burkholderia cepacia. These peptides may be used in various environments wherein antimicrobial treatment is desired, such as industrial and clinical settings. The peptides may be made in accordance with any appropriate method. The peptides of the present invention are characterized by specific properties as described below. These properties include, but are not limited to, hydrophobic, cationic and structural characteristics.

The peptides of the present invention possess activity toward microbes, especially Burkholderia cepacia, in which activity can be described as “antimicrobial”. As used herein, the term “antimicrobial” is meant to include prevention, inhibition or termination of a microbe. “Prevention” can be considered to be the obstruction or hindrance of any potential microbial growth. “Inhibition” can be considered to be a reduction in microbial growth. This may occur via, but is not limited to, a microbiostatic mechanism such as interference in the synthesis of the cell wall or binding to ribosomal subunits to prevent production of microbials proteins. “Termination” can be considered to be actual killing of the microbes by the presence of the composition. This may occur via, but is not limited to, a microbiocidal mechanism such as a change in osmotic pressure leading to bursting of the cell or formation of leaky channels in the cell wall and membrane causing loss of cellular material.

As used herein, “microbes” is meant to include any organism comprised of the phylogenetic domains bacteria and archaea, as well as unicellular and filamentous fungi (such as yeasts and molds), unicellular and filamentous algae, unicellular and multicellular parasites, and viruses.

The present invention is effective against bacteria including Gram-positive and Gram-negative cocci, Gram positive and Gram negative straight, curved and helical/vibroid and branched rods, sheathed bacteria, sulfur-oxidizing bacteria, sulfur or sulfate-reducing bacteria, spirochetes, actinomycetes and related genera, myxobacteria, mycoplasmas, rickettsias and chlamydias, cyanobacteria, archea, fungi, parasites, viruses and algae. More specifically, the present invention is useful against Burkholderia cepacia.

The Gram-positive and Gram-negative cocci include, but are not limited to, Aerococcus, Enterococcus, Halococcus, Leuconostoc, Micrococcus, Mobiluncus, Moraxella catarrhalis, Neisseria (including N. gonorrheae and N. meningitidis), Pediococcus, Peptostreptococcus, Staphylococcus species (including S. aureus, methicillin-resistant S. aureus, coagulase-negative S. aureus, and S. saprophyticus), Streptococcus species (including S. pyogenes, S. agalactiae, S. bovis, S. pneumoniae, S. mutans, S. sanguis, S. equi, S. equinus, S. thermophilus, S. morbillorum, S. hansenii, S. pleomorphus, and S. parvulus), and Veillonella.

The Gram-positive and Gram-negative straight, curved, helical/vibrioid and branched rods include, but are not limited to, Acetobacter, Acinetobacter, Actinobacillus equuli, Aeromonas, Agrobacterium, Alcaligenes, Aquaspirillum, Arcanobacterium haemolyticum, Bacillus species (including B. cereus and B. anthracis), Bacteroides species (including B. fragilis), Bartonella, Bordetella species (including B. pertussis), Brochothrix, Brucella, Burkholderia cepacia, Calymmatobacterium granulomatis, Campylobacter species (including C. jejuni), Capnocytophaga, Caulobacter, Chromobacterium violaceum, Citrobacter, Clostridium species (including C. perfringens, C. tetani and C. difficile), Comamonas, Curtobacterium, Edwardsiella, Eikenella, Enterobacter, Erwinia, Erysipelothrix, Escherichia species (including E. coli), Flavobacterium species (including F. meninosepticum), Francisella species (including F. tularensis), Fusobacterium (including F. nucleatum), Gardnerella species (including G. vaginalis), Gluconobacter, Haemophilus species (including H. influenzae and H. ducreyi), Hafnia, Helicobacter (including H.pylori), Herpetosiphon, Klebsiella species (including K. pneumoniae), Kluyvera, Lactobacillus, Legionella species (including L. pneumophila), Leptotrichia, Listeria species (including L. monocytogenes), Microbacterium, Morganella, Nitrobacter, Nitrosomonas, Pasteurella species (including P. multocida), Pectinatus, Porphyromonas gingivalis, Proteus species (including P. mirabilis), Providencia, Pseudomonas species (including P. aeruginosa, P. mallei, P. pseudomallei and P. solanacearum), Rahnella, Renibacterium salmoninarum, Salmonella, Serratia, Shigella, Spirillum, Streptobacillus species (including S. moniliformis), Vibrio species (including V. cholerae and V. vulnificus), Wolinella, Xanthobacter, Xenorhabdus, Yersinia species (including Y. pestis and Y. enterocolitica), Zanthomonas and Zymomonas.

The sheathed bacteria include, but are not limited to, Crenothrix, Leptothrix and Sphaerotilus. The sulfur-oxidizing bacteria include, but are not limited to, Beggiatoa, Gallionella, Sulfolobus, Thermothrix, Thiobacillus species (including T. ferroxidans), Thiomicrospira and Thiosphaera. The sulfur or sulfate-reducing bacteria include, but are not limited to, Desulfobacter, Desulfobulbus, Desulfococcus, Desulfomonas, Desulfosarcina, Desulfotomaculum, Desulfovibrio and Desulfuromonas.

The spirochetes include, but are not limited to, Treponema species (including T. pallidum, T. pertenue, T. hyodysenteriae and T. denticola), Borrelia species (including B. burgdorferi and B. recurrentis), Leptospira and Serpulina.

The actinomycetes and related genera include, but are not limited to, Acetobacterium, Actinomyces species (including A. israelii), Bifidobacterium, Brevibacterium, Corynebacterium species (including C. diphtheriae, C. insidiosum, C. michiganese, C. rathayi, C. sepedonicum, C. nebraskense), Dermatophilus, Eubacterium, Mycobacterium species (including M. tuberculosis and M. leprae), Nocardia, Propionibacterium, Rhodococcus and Streptomyces.

The myxobacteria include, but are not limited to, Chondromyces, Cystobacter, Melittangium, Myxococcus, Nannocystis, Polyangium and Stigmatella. The mycoplasmas include, but are not limited to, Mycoplasma species (including M. pneumoniae), Mycoplasma-like organisms of plants and invertebrates, Spiroplasma and Ureaplasma species (including U. urealyticum).

The rickettsias and chlamydias include, but are not limited to, Aegyptianella, Anaplasma, Chlamydia species (including C. pneumoniae, C. trachomatis and C. psittaci), Cowdria, Coxiella, Ehrlichia, Eperythrozoon, Haemobartonella, Neorickettsia, Rickettsia and Rickettsiella. The cyanobacteria include, but are not limited to, Anabaena, Nostoc, Oscillatoria, Pleurocapsa, Prochloron and Synechococcus.

The archea include, but are not limited to, all methanogens (Methanobacterium, Methanobrevibacter, Methanococcoides, Methanococcus, Methanogenium, Methanolobus, Methanomicrobium, Methanoplanus, Methanosarcina, Methanospirillum, Methanothermus and Methanothrix), and the genera Acidianus, Archaeoglobus, Desulfurococcus, Haloarcula, Halobacterium, Halococcus, Haloferax, Natronobacterium, Natronococcus, Pyrococcus, Pyrodictium, Staphylothermus, Sulfolobus, Thermococcus, Thermophila, Thermoplasma and Thermoproteus.

The present invention may also be used against fungi which include, but are not limited to, Acremonium, Aspergillus, Blastomyces species (including B. dermatitidis), Candida species (including C. albicans), Ceratocystis, Chaetomium, Coccidioides species (including C. immitis), Cryptococcus neoformans, Epidermophyton, Fusarium species (including F. oxysporum), Gongronella, Histoplasma species (including H. capsulatum), Hormonea, Malassezia furfur, Microsporum, Mycosphaerella fijiensis, Paracoccidiodes brasiliensis, Penicillium, Pneumocystis carinii, Pythium, Rhizoctonia, Rhodotorula, Saccharomyces, Sporothrix schenckii, Torula, Trichoderma, Trichophyton species (including T. mentagrophytes and T. rubrum) and Trichothecium.

The present invention may be used against parasites which include, but are not limited to, Acanthamoeba species, Ascaris lumbricoides, Babesia, Balamuthia, Balantidium, Blastocystis species including B. hominis, Chilomastix, Clonorchis sinensis, Cryptosporidium parvum, Cyclospora, Dientamoebafragilis, Diphyllobothrium, Echinococcus, Endolimax, Entamoeba species (including E. histolytica), Enterobius species (including E. vermicularis), Giardia lamblia, hookworms (including Necator, Ancylostoma, and Unicinaria), Hymenolepsis, Iodamoeba, Isospora, Leishmania, Mansonella, microsporidia, Microsporidium, Naegleria fowleri, Onchocerca, Plasmodium (including P. falciparum, P. vivax, P. ovale and P. malariae), Schistosoma (including S. haematobium and S. mansoni), Strongyloides species (including S. stercoralis), tapeworms (including Taenia species), Toxoplasma (including T. gondii), Trichinella (including T. spiralis), Trichomonas vaginalis, Trichuris species including T. trichiura, Trypanosoma, Dirofilaria, Brugia, Wuchereria, Vorticella, Eimeria species, Hexamita species and Histomonas meleagidis.

The present invention may also be used against viruses which include, but are not limited, to adenovirus, arborviruses (including hanta virus), astrovirus, coronavirus, cytomegalovirus, enteroviruses (including coxsackievirus A), Epstein-Barr virus, hepatitis A virus, hepatitis B virus, herpes viruses (including herpes simples virus or HSV), human immunodeficiency virus (HIV), human papilloma virus, human T-cell leukemia virus, influenza virus, mumps virus, Norwalk viruses, orbivirus, parainfluenzae viruses, parvovirus B19, poxviruses, Rabies virus, respiratory syncytial virus, rhinovirus, rotavirus, Rubella virus, varicella-zoster virus, vesicular stomatitis virus, cauliflower mosaic virus, cowpea mosaic virus, cowpox virus and rabbit myxomatis virus.

In addition, the present invention may be used against algae which include, but are not limited to, Chlorella, Fragilaria, Gomphonema, Navicula, Nitzschia, Pfiesteria (dinoflagellate), Scenedesmus, Skeletoneona and Ulothrix. The peptides of this invention are useful in the treatment of diseases caused by, but not limited to, bacteria, fungi, viruses and parasites in animals, plants, avian and aquatic organisms. For instance, diseases caused by gram-positive and/or gram-negative bacteria, and treatable with the present invention include abscesses, bacteremia, contamination of peritoneal dialysis fluid, endocarditis, pneumonia, meningitis, osteomyelitis, cellulitis, pharyngitis, otitis media, sinusitis, scarlet fever, arthritis, urinary tract infection, laryngotracheitis, erysipeloid, gas gangrene, tetanus, typhoid fever, acute gastroenteritis, bronchitis, epiglottitis, plague, sepsis, chancroid, wound and burn infection, cholera, glanders, periodontitis, genital infections, empyema, granuloma inguinale, Legionnaire's disease, paratyphoid, bacillary dysentary, brucellosis, diphtheria, pertussis, botulism, toxic shock syndrome, mastitis, rheumatic fever, cystic fibrosis, eye infections, plaque, and dental caries. Other uses include swine erysipelas, peritonitis, abortion, encephalitis, anthrax, nocardiosis, pericarditis, mycetoma, peptic ulcer, melioidosis, Haverhill fever, tularemia, Moko disease, galls (such as crown, cane and leaf), hairy root, bacterial rot, bacterial blight, bacterial brown spot, bacterial wilt, bacterial fin rot, dropsy, columnaris disease, pasteurellosis, furunculosis, enteric redmouth disease, vibriosis of fish, fouling of medical devices.

Peptides of the present invention may also be useful in treating diseases caused by spirochetes including syphilis, yaws, Lyme disease, Weil's disease, meningitis, leptospirosis, tick- and louse-borne relapsing fever, tick spirochetosis and canine, avian, rodent or lagomorph borreliosis. In addition, diseases caused by actinomycetes may be treatable by the present invention including tuberculosis, leprosy, cervicofacial lesions, abdominal lesions, thoracic lesions, pulmonary lesions and lesions of other organs, leafy gall and fish corynebacteriosis. Treatable rickettsial and chlamydial diseases or infections by the present invention include psittacosis, boutonneuse fever, ehrlichiosis, typhus fever, murine typhus, Brill's disease, Rocky Mountain spotted fever, Q fever, rickettsial pox, lymphogranuloma venereum, urethritis and trachoma. Treatable diseases or infections by mycoplasma include lethal yellowing.

Fungal infections treatable by the present invention include oral, cutaneous and vaginal thrush, cryptococcosis, superficial mycosis (including Athlete's foot), subcutaneous mycosis (including sporotrichosis), systemic mycosis (including histoplasmosis and coccidioidomycosis), Farmer's lung, aflatoxin disease, histoplasmosis, pneumonia, endocardititis, burn infections, mucormycosis, pityriasis versicolor, fungemia due to indwelling catheter infections, damping off, rot, panama disease, black leaf streak, anthracnose, apple scab, black knot, rust, canker, gray mold, blue mold, blight, powdery and downy mildew, wilt, damping off and leaf spot.

Viral infections treatable by the present invention include common colds, hemorrhagic fevers, mononucleosis, genital disease, keratoconjunctivitis, encephalitis, neonatal HSV, mucocutaneous HSV, chicken pox, retinitis, AIDS, influenza, pneumonia, bronchiolitis, genital papilloma, measles (including German measles), rabies, rubella, mumps, shingles, poliomyelitis, viral diarrhea, yellow fever, zoster, roseola, laryngotracheobronchitis, gastroenteritis, hepatitis (including hepatitis A and B), dengue fever, orf virus infection, molluscum contagiosum virus infection, fruit and vegetable mosaic viruses, tobacco ringspot virus, leaf curl virus, dropsy, cauliflower disease and necrotic viruses of fish.

Parasitic infections treatable by the present invention include trichinosis, schistosomiasis, malaria, giardiasis, amoebiasis, encephalitis, keratitis, gastroenteritis, urogenital infections, toxoplasmosis, African sleeping sickness, white spot disease, slimy skin disease, chilodonella, costia, hexamitiasis, velvet and coral fish disease.

Peptides of the present invention are also useful as infection or inflammation seeking agents or as T-cell activators.

More preferably, peptides of the present invention are useful in the treatment of infections in respiratory disorders including cystic fibrosis, pneumonia or bacterial bronchitis.

The peptide sequences may be selected from synthetic combinatorial libraries using methods known to one of ordinary skill in the art to produce a mixture of peptides or a single peptide within a mixture with optimal activity for a target application. The peptide mixtures may be selected from an L-hexapeptide library comprised of equimolar concentrations of all peptides. The amino acids comprising the peptides are selected from all of the naturally occurring amino acids, as well as non-natural amino acids.

The standard three letter and single letter codes for amino acids are used herein and are as follows:

| | | | | | | | | |
|---|---|---|---|---|---|---|---|---|
| Ala | (A) | Alanine | Cys | (C) | Cysteine | Asp | (D) | Aspartic acid |
| Glu | (E) | Glutamic acid | Phe | (F) | Phenylalanine | Gly | (G) | Glycine |
| His | (H) | Histidine | Ile | (I) | Isoleucine | Lys | (K) | Lysine |
| Leu | (L) | Leucine | Met | (M) | Methionine | Asn | (N) | Asparagine |
| Pro | (P) | Proline | Gln | (Q) | Glutamine | Arg | (R) | Arginine |
| Ser | (S) | Serine | Thr | (T) | Threonine | Val | (V) | Valine |
| Trp | (W) | Tryptophan | Tyr | (Y) | Tyrosine | | | |

The present invention is useful in a variety of environments including industrial, clinical, the household, and personal care. The peptide compositions of the present invention for industrial, pharmaceutical, household and personal care use may comprise at least one active ingredient, of which the peptide mixture of the present invention is an active ingredient acting alone, additively, or synergistically against the target microbe.

The peptide mixtures of this invention may be delivered in a form suitable for use in environments including industry, pharmaceutics, household, and personal care. The peptides of the present invention are preferably soluble in water and may be applied or delivered with an acceptable carrier system. The composition may be applied or delivered with a suitable carrier system such that the active ingredient may be dispersed or dissolved in a stable manner so that the active ingredient, when it is administered directly or indirectly, is present in a form in which it is available in a particularly advantageous way.

Also, the separate components of the peptide compositions of the present invention may be preblended or each component may be added separately to the same environment according to a predetermined dosage for the purpose of achieving the desired concentration level of the treatment components and so long as the components eventually come into intimate admixture with each other. Further, the present invention may be administered or delivered on a continuous or intermittent basis.

The peptide mixtures of the present invention, when present in a composition will preferably be present in an amount of about 0.000001% to about 100%, more preferably from about 0.001% to about 50%, and most preferably from about 0.01% to about 25%.

For compositions of the present invention comprising peptide mixtures of the present invention, when a carrier is present, the composition comprises preferably from about 50% to about 99%, more preferably from about 25% to about 99%, and most preferably from about 1% to about 99% by weight of at least one carrier.

The present invention and any suitable carrier may be prepared for delivery in forms including solution, microemulsion, suspension or aerosol. Generation of the aerosol or any other means of delivery of the present invention may be accomplished by any of the methods known in the art. For example, in the case of aerosol delivery, the antimicrobial composition is supplied in a finely divided form along with any suitable carrier with a propellant. Liquified propellants are typically gases at ambient conditions and are condensed under pressure. The propellant may be any acceptable and known in the art including propane and butane, or other lower alkanes, such as those of up to 5 carbons. The antimicrobial composition is held within a container with an appropriate propellant and valve, and maintained at elevated pressure until released by action of the valve.

The compositions may be prepared in a conventional form suitable for, but not limited to topical or local application such as an ointment, paste, gel, spray and liquid, by including stabilizers, penetrants and the carrier or diluent with peptide according to a known technique in the art. These preparations may be prepared in a conventional form suitable for enteral, parenteral, topical or inhalational applications.

The present invention may be used in compositions suitable for household use. For example, compositions of the present invention are also useful as an active antimicrobial ingredient in household products such as cleansers, detergents, astringents, disinfectants, dishwashing liquids, and soaps. The antimicrobial composition of the present invention may be delivered in an amount and form effective for the prevention, removal or termination of microbes.

The antimicrobial composition for household use may be defined as comprising at least one peptide mixture of the present application and at least one suitable carrier. Preferably, the composition comprises from about 0.00001% to about 50%, more preferably from about 0.0001% to about 25%, most preferably from about 0.0005% to about 10% by weight of peptide mixture based on the weight percentage of the total composition.

The present invention may further be used in hygiene compositions for personal care. For instance, compositions of the present invention are useful as an active ingredient in personal care products such as facial cleansers, astringents, body wash, shampoos, conditioners, cosmetics and other hygiene products. The hygiene composition may comprise any carrier or vehicle known in the art to obtain the desired form (such as solid, liquid, semisolid or aerosol) as long as the effects of the peptide mixture of the present invention are not impaired. Methods of preparation of hygiene compositions are not described herein in detail, but are known in the art. For its discussion of such methods, THE CTFA COSMETIC INGREDIENT HANDBOOK, Second Edition, 1992, and pages 5-484 of A FORMULARY OF COSMETIC PREPARATIONS (Vol. 2, Chapters 7-16) are incorporated herein by reference.

The hygiene composition for use in personal care may be defined as comprising at least one peptide mixture of the present application and at least one suitable carrier. Preferably, the composition comprises from about 0.00001% to about 50%, more preferably from about 0.0001% to about 25%, most preferably from about 0.0005% to about 10% by weight of peptide mixture based on the weight percentage of the total composition.

The peptide mixtures of the present invention may be used in industry. In the industrial setting, the presence of microbes can be problematic, as microbes are often responsible for industrial contamination and biofouling. Antimicrobial compositions for industrial applications comprise an effective amount of the peptide mixtures of the present invention in an antimicrobial composition for industrial use with at least one acceptable carrier or vehicle known in the art to be useful in the treatment of such systems. Such carriers or vehicles may include diluents, deflocculating agents, penetrants, spreading agents, surfactants, suspending agents, wetting agents, stabilizing agents, compatability agents, sticking agents, waxes, oils, co-solvents, coupling agents, foams, antifoaming agents, natural or synthetic polymers, elastomers and synergists. Methods of preparation, delivery systems and carriers for such antimicrobial compositions are not described here in detail, but are known in the art. For its discussion of such methods, U.S. Pat. No. 5,939,086 is herein incorporated by reference. Furthermore, the preferred amount of antimicrobial composition to be used may vary according to the peptide mixture and situation in which the composition is being applied.

The antimicrobial compositions of the present invention may be useful in nonaqueous environments. Such nonaqueous environments may include, but are not limited to, terrestrial environments, dry surfaces or semi-dry surfaces in which the antimicrobial composition is applied in a manner and amount suitable for the situation. The antimicrobial compositions of the present invention may also be used to form contact-killing coatings or layers on a variety of substrates including personal care products (such as toothbrushes, contact lens cases and dental equipment), healthcare products, household products, food preparation surfaces and packaging, and laboratory and scientific equipment. Further, other substrates include medical devices such as catheters, urological devices, blood collection and transfer devices, tracheotomy devices, intraocular lenses, wound dressings, sutures, surgical staples, membranes, shunts, gloves, tissue patches, prosthetic devices (e.g., heart valves) and wound drainage tubes. Still further, other substrates include textile products such as carpets and fabrics, paints and joint cement.

The peptides may also be incorporated into polymers, such as polysaccharides (cellulose, cellulose derivatives, starch, pectins, alginate, chitin, guar, carrageenan), glycol polymers, polyesters, polyurethanes, polyacrylates, polyacrylonitrile, polyamides (e.g., nylons), polyolefins, polystyrenes, vinyl polymers, polypropylene silks or biopolymers. The peptides may be conjugated to any polymeric material with the following specified functionality: 1) carboxy acid, 2) amino group, 3) hydroxyl group and/or 4) haloalkyl group.

The antimicrobial composition for treatment of nonaqueous environments may be defined as comprising at least one peptide mixture of the present application and at least one suitable carrier. Preferably, the composition comprises from about 0.001% to about 75%, more preferably from about 0.01 to about 50%, most preferably from about 0.1% to about 25% by weight of peptide mixture based on the weight percentage of the total composition. The antimicrobial compositions of the present invention may be useful in aqueous environments. “Aqueous environments” as used herein, is meant to include any type of system containing water, including but not limited to, natural bodies of water such as lakes or ponds; artificial, recreational bodies of water such as swimming pools; and drinking reservoirs such as wells. The antimicrobial compositions of the present invention are useful in treating microbial growth in these aqueous environments and may be applied at or near the surface of water.

The antimicrobial composition for treatment of aqueous environments may be defined as comprising at least one peptide mixture of the present application and at least one suitable carrier. Preferably, the composition comprises from about 0.001% to about 50%, more preferably from about 0.003% to about 15%, most preferably from about 0.01% to about 5% by weight of peptide mixture based on the weight percentage of the total composition.

The composition of the present invention may be administered for clinical use, in a therapeutically effective amount and composition, to beings infected with a microorganism discussed above. Beings treatable clinically include all land, air and water animals, and plants, but preferably mammals and most preferably humans. Alternatively, the composition may be administered prophylactically. The therapeutic and prophylactic dose for the present invention may vary according to several factors including the age, weight, and condition of the individual, route of administration and/or other drug interactions. The principles and factors for determining dosage are not discussed here in detail, but are known in the art and may be referenced in pages 1-83 of GOODMAN AND GILMAN'S THE PHARMACOLOGICAL BASIS OF THERAPEUTICS (8th Edition). The preferred doses for therapeutic and prophylactic treatment may vary and can be adjusted to suit the individual and situation.

The therapeutically and prophylactically effective amount is preferably from about 0.5 mg/kg to about 100 mg/kg, more preferably from about 1 mg/kg to about 20 mg/kg, and most preferably from about 2 mg/kg to about 10 mg/kg.

In addition to the foregoing, the present invention also provides a process for the production of a pharmaceutical composition. Such process comprises bringing at least one of the individual components described thereof into intimate admixture with a peptide mixture of the present invention, and when required, compounding the obtained composition in unit dosage form, for example filling said composition into gelatin, e.g., soft or hard gelatin, capsules. Methods of preparation of pharmaceutical compositions are not described here in detail, but are known in the art. For its discussion of such methods, pages 1435-1694 of REMINGTON'S PHARMACEUTICAL SCIENCES (Part 8) is incorporated herein by reference.

The pharmaceutical composition may be defined as comprising at least one peptide mixture of the present application and at least one suitable carrier. Preferably, the composition comprises from about 0.000001% to about 75%, more preferably from about 0.00001% to about 25%, most preferably from about 0.0001% to about 12% by weight of peptide mixture based on the weight percentage of the total composition.

The pharmaceutical composition may be administered for treatment of any land, air or water animal potentially having or having at least one microbial infection. Treatment of an animal with the present invention may also include prophylactic treatment. The mode of administration is such as to deliver a binding inhibiting effective amount of the pharmaceutical composition to the site of infection. For example, therapeutic delivery of the pharmaceutical composition may be achieved via enteral administration, which includes oral, sublingual and rectal administration, or via parenteral administration, which includes intramuscular, intravenous and subcutaneous administration. Alternatively, therapeutic delivery of the pharmaceutical composition may also be achieved via other routes including topical and inhalational. As discussed above, preferred dosage ranges will vary according to the individual and situation.

Enteral administration of the pharmaceutical composition is preferably administered at a dosage of from about 0.01 mg/kg to about 100 mg/kg, more preferably from about 2 mg/kg to about 50 mg/kg, and most preferably from about 5 mg/kg to about 30 mg/kg.

Parenteral administration of the pharmaceutical composition is preferably administered at a dosage from about 0.01 mg/kg to about 100 mg/kg, more preferably from about 1 mg/kg to about 30 mg/kg, and most preferably from about 5 mg/kg to about 25 mg/kg.

Topical administration of the pharmaceutical composition is preferably administered at a dosage from about 0.000001% to about 20%, more preferably from about 0.001% to about 15%, and most preferably from about 0.025% to about 10%.

Inhalational administration of the pharmaceutical composition is preferably administered at a dosage from about 0.0001 mg to about 25 mg, more preferably from about 0.01 mg to about 15 mg, and most preferably from about 0.1 mg to about 10 mg.

The peptide mixtures of this invention may be delivered in a pharmaceutically acceptable composition suitable for any of the routes of administration discussed above. “Pharmaceutically acceptable” is used herein to refer to those materials which are within the scope of sound medical judgement, suitable for use in contact with the tissue of humans and lower animals, avian and aquatic organisms without undue toxicity, irritation, allergic response and the like commensurate with a reasonable benefit/risk ratio, and effective for their intended use in the composition.

The pharmaceutical compositions may include, but are not limited to, at least one acceptable carrier. The carrier is generally an inert bulk agent added to make the active ingredients easier to handle and can be solid, semisolid or liquid in the usual manner as well as understood in the art. Such a carrier may be a solvent, diluent or carrier comprising of waxes, cellulose derivatives, mineral oils, vegetable oils, petroleum derivatives, water, anhydrous lanolin, white petrolatum, liquid petrolatum, olive oil, ethanol and ethanol-polysorbate 80 solutions, propylene glycol-water solutions, and jojoba oils, methylcellulose or paraffin, beeswax, glyceryl stearate, PEG-2 stearate, propylene glycol stearate, glycol stearate, cetyl alcohol, stearyl alcohol, and any mixture thereof. Carriers used may include commercially available carriers or vehicles including Aquaphor® ointment base (Beirsdorf Inc.), Eucerin® creme/lotion (Beirsdorf), Acid Mantle® (Sandoz), Nutraderm® creme/lotion (Owen), Vehicle/N® or Vehicle/N® Mild (Neutrogena).

Pharmaceutical compositions of the invention may also include any delivery vehicle or device known in the art to enhance the transport of peptides across tissue and/or cell surfaces to reach the circulatory system and/or target site. Such delivery vehicles or devices may include liposomes or immunogenic liposomes, which may be adminstered in admixture with any carrier (discussed above) with regard to the intended route of administration, and standard pharmaceutical practice. Dosages of peptide mixtures associated with such delivery vehicles or devices will vary according to certain factors including the age, weight, and condition of the individual, as well as the pharmacokinetics and release characteristics of the peptides from the delivery vehicles or devices. Further, the ratio of peptide mixture to liposome and carrier will depend on the chemical nature, solubility, trapping efficiency, and stability of the peptides, as well as the dosage anticipated. Maximal delivery of the peptides of the present invention may be accomplished by varying the lipid:peptide ratio as well as the type of peptide and liposome used.

The present invention also provides a process for the production of an antibiofouling composition for industrial use. Such process comprises bringing at least one of any industrially acceptable carrier known in the art into intimate admixture with a peptide mixture of the present invention. The carrier may be any suitable carrier discussed above or known in the art.

The suitable antibiofouling compositions may be in any acceptable form for delivery of the composition to a site potentially having, or having, at least one living microbe. The antibiofouling compositions may be delivered with at least one suitably selected carrier as hereinbefore discussed using standard formulations. The mode of delivery may be such as to have a binding inhibiting effective amount of the antibiofouling composition at a site potentially having, or having at least one living microbe. The antibiofouling compositions of the present invention are useful in treating microbial growth that contributes to biofouling, such as scum or slime formation, in these aqueous environments. Examples of industrial processes in which these compounds might be effective include cooling water systems, reverse osmosis membranes, pulp and paper systems, air washer systems and the food processing industry. The antibiofouling composition may be delivered in an amount and form effective for the prevention, removal or termination of microbes.

The antibiofouling composition of the present invention preferably comprises at least one peptide mixture of the present application from about 0.001% to about 50%, more preferably from about 0.003% to about 15%, most preferably from about 0.01% to about 5% by weight of peptide mixture based on the weight percentage of the total composition.

The amount of antibiofouling composition is preferably delivered in an amount of about 1 mg/l to about 1000 mg/l, more preferably from about 2 mg/l to about 500 mg/l, and most preferably from about 20 mg/l to about 140 mg/l.

The peptides of the present invention may be modified at the N- and/or C-terminus. “Modifications” as used herein include modifications at the N-terminus and/or C-terminus or modification of any position on at least one amino acid residue. The modified peptides may be represented by Formulae I and II:

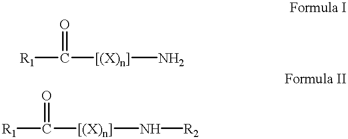

wherein:

X represents any of the natural or non-natural, modified or unmodified amino acids except glutamate (Glu) or aspartate (Asp);

n=1 to 10;

R₁ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₅-C₂₀ cycloalkylalkyl; C₄-C₂₀ alkenyloxyalkyl; C₄-C₂₀ alkynyloxyalkyl; C₄-C₂₀ (cycloalkyl)oxyalkyl; C₄-C₂₀ alkenylthioalkyl; C₄-C₂₀ alkynylthioalkyl; C₆-C₂₀ (cycloalkyl)thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;

R₂ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₅-C₂₀ cycloalkylalkyl; C₄-C₂₀ alkenyloxyalkyl; C₄-C₂₀ alkynyloxyalkyl; C₄-C₂₀ (cycloalkyl)oxyalkyl; C₄-C₂₀ alkenylthioalkyl; C₄-C₂₀ alkynylthioalkyl; C₆-C₂₀ (cycloalkyl)thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;

R₃ is independently hydrogen; C₁-C₄ alkyl; or phenyl optionally substituted with at least one R₈;

R₄ is independently hydrogen; C₁-C₈ alkyl; or phenyl optionally substituted with at least one R₈;

R₅ is independently C₁-C₆ alkyl; C₁-C₆ alkoxy; C₁-C₆ haloalkyl; halogen; C₂-C₈ alkynyl; C₁-C₆ thioalkyl; phenyl or phenoxy each optionally substituted with at least one R₈; cyano; nitro; C₁-C₆ haloalkoxy; C₁-C₆ haloalkythio; C₂-C₆ alkenyl; C₂-C₆ haloalkenyl; acetyl; CO₂CH₃; or N(C₁-C₂ alkyl)₂;

R₆ is independently methyl; ethyl; methoxy; methylthio; halogen; or trifluoromethyl;

R₇ is independently halogen; and

R₈ is independently halogen; C₁-C₄ alkyl; C₁-C₄ alkoxy; C₁-C₄ haloalkyl; nitro; or cyano.

As used herein, “hydrocarbyl” is defined by R₁ and R₂.

In the above recitations, the term “alkyl”, used either alone or in compound words such as “alkylthio,” “haloalkyl,” or “alkylthioalkyl” denotes straight-chain or branched alkyl; e.g., methyl, ethyl, n-propyl, i-propyl, or the different butyl, pentyl, hexyl, etc. isomers.

“Cycloalkyl” denotes cyclopropyl, cyclobutyl, cyclopentyl, and cyclohexyl. The term “cycloalkyloxyalkyl” denotes the cycloalkyl groups linked through an oxygen atom to an alkyl chain. Examples include cyclopentyloxymethyl and cyclohexyloxybutyl. The term “cycloalkylthioalkyl” are the cycloalkyl groups linked through a sulfur atom to an alkyl chain; e.g., cyclopropylthiopentyl. “Cycloalkylalkyl” denotes a cycloalkyl ring attached to a branched or straight-chain alkyl; e.g., cyclopropylmethyl and cyclohexylbutyl.

“Cycloalkylalkyl” denotes a cycloalkyl ring attached to a branched or straight-chain alkyl; e.g., cyclopropylmethyl and cyclohexylbutyl.

“Alkenyl” denotes straight chain or branched alkenes; e.g., 1-propenyl, 2-propenyl, 3-propenyl and the different butenyl, pentenyl, hexenyl, etc. isomers. Alkenyl also denotes polyenes such as 1,3-hexadiene and 2,4,6-heptatriene.

“Alkynyl” denotes straight chain or branched alkynes; e.g., ethynyl, 1-propynyl, 3-propynyl and the different butynyl, pentynyl, hexynyl, etc. isomers. “Alkynyl” can also denote moieties comprised of multiple triple bonds; e.g., 2,7-octadiyne and 2,5,8-decatriyne.

“Alkoxy” denotes methoxy, ethoxy, n-propyloxy, isopropyloxy and the different butoxy, pentoxy, hexyloxy, etc. isomers. “Alkoxyalkenyl” and “alkoxyalkynyl” denoted groups in which the alkoxy group is bonded through the oxygen atom to an alkenyl or alkynyl group, respectively. Examples include CH₃OCH₂CH≡CH and (CH₃)₀CHOCH₂C≡CCH₂. The corresponding sulfur derivatives are denoted “alkylthioalkenyl” and “alkylthioalkynyl. Examples of the former include CH₃SCH₂CH═CH and CH₃CH₂SCH₂(CH₃)CH═CHCH₂, and an example of the latter is CH₃CH₂CH₂CH₂SCH₂C≡C.

“Alkenyloxy” denotes straight chain or branched alkenyloxy moieties. Examples of alkenyloxy include H₂C═CHCH₂O, (CH₃)₂C═CHCH₂O, (CH₃)CH═CHCH₂O, (CH₃)CH═C(CH₃)CH₂O and CH₂═CHCH₂CH₂O. “Alkenylthio” denotes the similar groups wherein the oxygen atom is replaced with a sulfur atom; e.g., H₂C═CHCH₂S and (CH₃)CH═C(CH₃)CH₂S. The term “alkenyloxyalkyl” denotes groups in which the alkenyloxy moiety is attached to an alkyl group. Examples include H₂C═CHCH₂OCH₂CH₂, H₂C═CHCH₂OCH(CH₃)CH₂, etc. “Alkenylthioalkyl” denotes the alkenylthio moieties bonded to an alkyl group. Examples include H₂C═CHCH₂SCH(CH₃)CH(CH₃) and (CH₃)CH═C(CH₃)CH₂SCH₂.

“Alkynyloxy” denotes straight or branched alkynyloxy moieties. Examples include HC≡CCH₂O, CH₃C≡CCH₂O and CH₃C≡CCH₂CH₂O. “Alkynyloxyalkyl” denotes alkynyloxy moieties bonded to alkyl groups; e.g., CH₃C≡CCH₂OCH₂CH₂ and HC≡CCH₂OCH(CH₃)CH₂. “Alkynylthioalkyl” denotes alkynylthio moieties bonded to alkyl groups. Example include CH₃C CCH₂SCH₂CH₂ and CH₃C≡CCH₂CH₂SCH(CH₃)CH₂.

“Alkylthio” denotes methylthio, ethylthio, and the different propylthio, butylthio, pentylthio and hexylthio isomers. “Alkylthioalkyl” denotes alkylthio groups attached to an alkyl chain; e.g., CH₃CH₂SCH₂CH(CH₃) and (CH₃)₂CHSCH₂.

“Alkylsulfinyl” denotes both enantiomers of an alkylsulfinyl group. For example, CH₃S(O), CH₃CH₂S(O), CH₃CH₂CH₂S(O), (CH₃)₂CHS(O) and the different butylsulfinyl, pentylsulfinyl and hexylsufinyl isomers. “Alkylsulfinylalkyl” denotes alkylsulfinyl groups attached to an alkyl chain; e.g., CH₃CH₂S(O)CH₂CH(CH₃) and (CH₃)₂CHS(O)CH₂.

Examples of “alkylsulfonyl” include CH₃S(O)₂, CH₃CH₂S(O)₂, CH₃CH₂CH₂S(O)₂, (CH₃)₂CHS(O)₂ and the different butylsulfonyl, pentylsulfonyl and hexylsulfonyl isomers. “Alkylsulfonylalkyl” denotes alkylsulfonyl groups attached to an alkyl chain; e.g., CH₃CH₂S(O)₂CH₂CH(CH₃) and (CH₃)₂CHS(O)₂CH₂.

The term “halogen”, either alone or in compound words such as “haloalkyl”, denotes fluorine, chlorine, bromine or iodine. Further, when used in compound words such as “haloalkyl”, said alkyl may be partially or fully substituted with halogen atoms which may be the same or different. Examples of “haloalkyl” include F₃C, ClCH₂, CF₃CH₂ and CF₃CF₂. Examples of “haloalkenyl” include (Cl)₂C≡CHCH₂ and CF₃CH₂CH=CHCH₂. “Haloalkenyloxyalkyl” denotes haloalkenyl groups bonded to oxygen and in turn bonded to alkyl groups. Examples include CF₃CH₂CH═CHCH₂OCH₂ and (Cl)₂C═CHCH₂OCH₂CH₂. Examples of “haloalkynyl” include HC≡CCHCl, CF₃C≡C, CCl₃C≡C and FCH₂C≡CCH₂. “Haloalkynyloxyalkyl” denotes haloalkynyl groups bonded through an oxygen atom to an alkyl moiety. Examples include CF₃C≡CCH₂OCH₂CH₂, ClCH₂C≡CCH₂CH₂OCH(CH₃), etc. Examples of “haloalkoxy” include CF₃O, CCl₃CH₂O, CF₂HCH₂CH₂O and CF₃CH₂O. “Haloalkoxyalkyl” denotes haloalkoxy groups bonded to straight-chain or branched alkyl groups; e.g., CF₂HCH₂CH₂OCH₂CH₂, CCl₃CH₂OCH(CH₃) and CF₃OCH₂.

“Trialkylsilyl” designates a group with three alkyl groups bonded to silicon; e.g., (CH₃)₃Si and t-Bu(CH₃)₂Si. “Trialkylsilylalkyl” denotes trialkylsilyl groups bonded to another straight-chain or branched alkyl group. Examples include (CH₃)₃SiCH₂ and t-Bu(CH₃)₂SiCH₂CH(CH₃)CH₂. The total number of carbon atoms in a substituent group is indicated by the “C₁-Cⱼ” prefix where i and j are numbers from 1 to 10. For example, C₁-C₃ alkylsulfonyl designates methylsulfonyl through propylsulfonyl; C₂ alkoxyalkoxy designates CH₃OCH₂O; C₃ alkoxyalkoxy designates, for example, CH₃OCH₂CH₂O or CH₃CH₂OCH₂O; and C₄ alkoxyalkoxy designates the various isomers of an alkoxy group substituted with a second alkoxy group containing a total of 4 carbon atoms, examples including CH₃CH₂CH₂OCH₂O, and CH₃CH₂OCH₂CH₂O. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂.

Amino acid chains are from N-terminus to C-terminus. Furthermore, in the formulae, the R₁(C═O)— group is bound to the alpha nitrogen of the N-terminal amino acid of the peptide. The —NH₂ group (Formula I) or the —NH—R₂ group (Formula II) is bound to the carbon of the alpha carboxyl group of the C-terminal amino acid.

Preferably R₁ comprises from about 5 to about 15 carbon atoms, and more preferably comprises from about 6 to about 11 carbon atoms. Preferably R₁ comprises an alkyl group having from about 1 to about 20 carbon atoms. Preferably the alkyl group comprises from about 5 to about 15 carbon atoms, and more preferably comprises from about 6 to about 11 carbon atoms.

Preferably R₂ comprises 5 to 15 carbon atoms, and more preferably from about 6 to about 11 carbon atoms. Preferably, R₂ comprises hydrogen, or R₂ comprises an alkyl group. When R₂ is an alkyl group, preferably R₂ comprises from about 5 to about 15 carbon atoms, and more preferably from about 6 to about 11 carbon atoms.

Peptides of the present invention may comprise residues from any of the 20 natural amino acids. These natural amino acids may be in the D or L configuration. The terms D and L are used herein as they are known to be used in the art. In addition, modified peptides of the present invention may also comprise a monomer or dimer.

The amino acids of the peptides of the present invention may also be modified. The carboxyl group on the C-terminal end of the peptide may be esterified with an alkyl, substituted alkyl, alkene, substituted alkene, alkyne, substituted alkyne or with an aryl group (including heterocycles and polynuclear aromatic compounds). Carboxyl groups may be amidated. Carboxyl groups may also be reduced to alcohols, and potentially further converted to alkyl or alkyl halide ethers. Amino groups may be acylated, alkylated or arylated. Benzyl groups may be halogenated, nitrosylated, alkylated, sulfonated or acylated. These modifications are meant to be illustrative and not comprehensive of the types of modifications possible. Modification of the amino acids would likely add to the cost of synthesis and therefore is not preferred.

The present invention comprises mixtures containing peptides with antimicrobial activity. Peptide mixtures of the present invention may be selected from an L-hexapeptide library synthesized using the 20 natural amino acids and comprised of equimolar concentrations of all potential combinations of hexapeptides. The hexapeptides are represented by D₁D₂U₃U₄U₅U₆, and may be N- and/or C-terminally modified as described above. Each peptide mixture consists of all combinations of hexapeptides wherein D₁ and D₂ comprise defined amino acids, and U₃, U₄, U₅ and U₆ are undefined amino acids. Thus, there are 400 mixtures in each hexapeptide library, each consisting of the 160,000 sequences represented by a defined pair of amino acids as D₁D₂, and all possible combinations as U₃U₄U₅U₆. A single pure peptide demonstrating activity represents 0.000625% by weight of the total weight of the peptide mixture.

Preferred amino acids for D₁ position are arginine (Arg), lysine (Lys), methionine (Met), serine (Ser), threonine (Thr) or tryptophan (Trp).

Preferred amino acids for D₂ position are arginine (Arg), histidine (His), cysteine (Cys), threonine (Thr), tyrosine (Tyr) or tryptophan (Trp).

Still more preferred are the hexapeptide sequences wherein the first two amino acids (D₁D₂) comprise Arg-Tyr, Arg-Cys, Arg-Trp, Ser-Thr, Met-Trp, Lys-Trp, Thr-Trp, Trp-Arg, Trp-His, Trp-Tyr and Trp-Trp.

Most preferred are the hexapeptide sequences in which the first two amino acids are (D₁D₂) are Thr-Trp (Thr-Trp-U₃U₄U₅U₆).

The amino acids in positions U₃, U₄, U₅ or U₆ may consist of any of the natural amino acids.

The peptide of the present invention may be synthesized by solid-phase synthesis as described originally by Merrifield in pages 2149-2154 of J. Amer. Chem. Soc., vol. 85, 1963, and may be modified according to Peptides: synthesis, structures and applications, Gutte B. (ed.), Academic Press, NY, 1995, and Chemical approaches to the synthesis of peptides and proteins, Lloyd-Williams P., Alberico F., Giralt E. (eds.), CRC Press, NY, 1997. Generally, the C-terminal amino acid (with protected N-terminus) is attached to an appropriate solid support via the α-carboxyl group. The N-terminus is protected by an appropriate protecting group (such as tert-butyloxycarbonyl [Boc] or 9-fluorenylmethoxycarbonyl [Fmoc]). An example of a resin is a copolymer of styrene and 1% divinylbenzene. The N α-protecting group is removed, and the amino acid that is N-terminal to the attached amino acid is coupled to the attached amino acid using appropriate coupling reagents (such as dicyclohexylcarbodiimide). The peptide is elongated by repeating the deprotection and coupling steps. When all of the amino acids have been added, side-chain protecting groups used during the synthesis are removed, and the peptide is cleaved from the resin. An acyl chain may be attached by a condensation reaction with the N α-amide of the N-terminal amino acid of a peptide or to the C-terminal amide of the peptide. The acyl chain is added after removal of the Fmoc-group and prior to side chain deprotection. Acetic anhydride may also be used for N-terminal acetylation. For a C-terminal amide, an appropriate amide-containing resin is chosen such that when the peptide is cleaved from the resin, the amide group is retained on the peptide. Common solid supports for the synthesis of peptide amides are benzhydrylamide derivatives, such as 4-methylbenzhydrylamine resin. The peptide amide can be cleaved from the resin using hydrogen fluoride.

The peptides can be synthesized individually using a parallel synthesis approach, such as the tea bag method of simultaneously synthesizing equimolar amounts of multiple peptides as described in U.S. Pat. No. 5,504,190. Other methods of solid-phase synthesis known in the art may also be used to synthesize the peptides of the present invention.

Without further elaboration, it is believed that one skilled in the art can, using the preceding description, utilize the present invention to its fullest extent.

The following provides examples of the invention. Examples 1-2 are actual examples; Examples 3-15 are prophetic. These examples are merely illustrative of the invention and are not intended to limit the scope of the disclosure or any claim.

### EXAMPLES

### Example 1

### Materials and Methods of Peptide Synthesis and Bacterial Assays

Synthesis of Peptides

The peptides of the present invention were synthesized via solid-phase synthesis by Multiple Peptide Systems (San Diego, Calif.) according to the above methods. However, the peptides of the present invention may also be synthesized by any known method in the art.

Antimicrobial Assay

Cultures of Burkholderia cepacia (ATCC 25416) were grown (30° C.) in 0.5× mTGE Broth (Difco; Detroit, Mich.) for 19 h in an incubator shaker (200 rpm; Model G-25, New Brunswick Scientific, Edison, N.J.). The cultures were subjected to centrifugation (20 min, 22C, 2890× g, Labofuge A, American Scientific Products, Houston, Tex.) and resuspension in Wilson's Salts solution. Wilson's Salts solution (pH 7.0) contains (g/l): K₂HPO₄, 3.0; KH₂PO₄, 1.5; MgSO₄.7 H₂O, 0.1; (NH₄)₂SO₄, 1.0. The assays were performed in 96-well “U”-bottom microtiter plates (Dynatech Laboratories, Inc., Chantilly, Va.) in a total volume of 100 μl. The assay mixture (final concentration) consisted of 0.25× mTGE, peptide at 625 ppm, and inoculum (2.5×10⁵ cells/ml). The plates were incubated for 18 h at 30° C., and growth of the organisms was determined by measuring the change in optical density at 540 nm (Spectramax 250, Molecular Devices, Sunnyvale, Calif.).

### Example 2

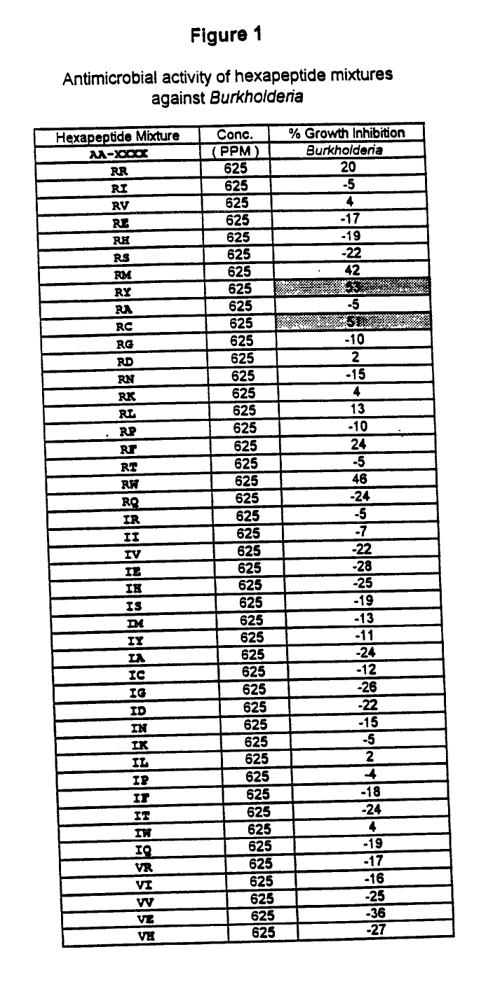

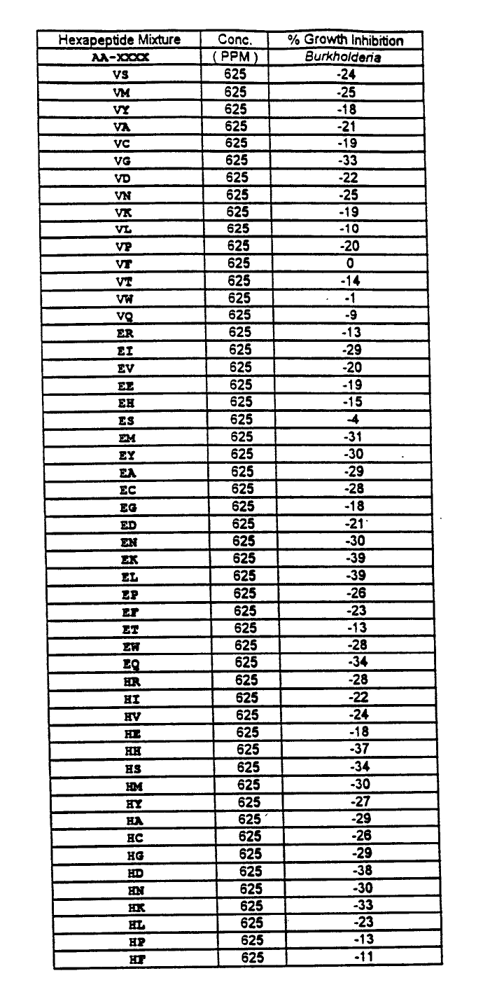

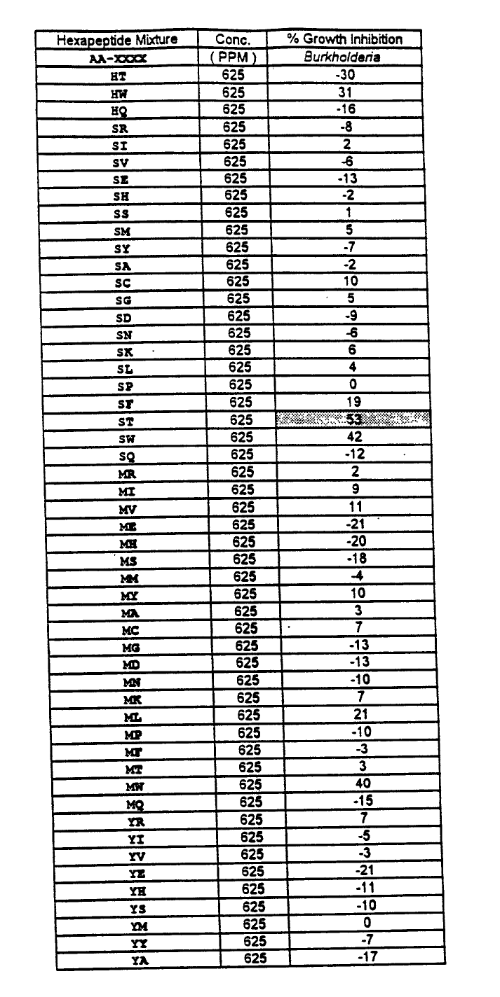

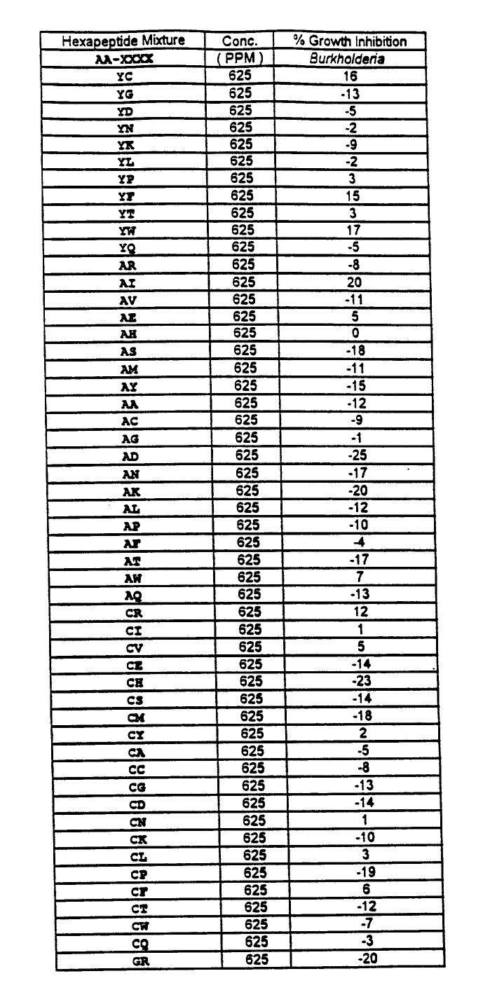

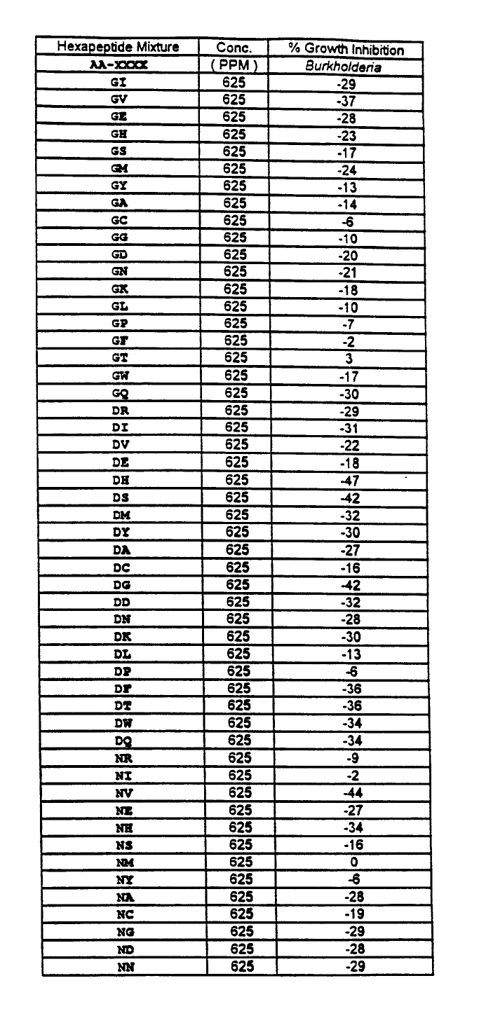

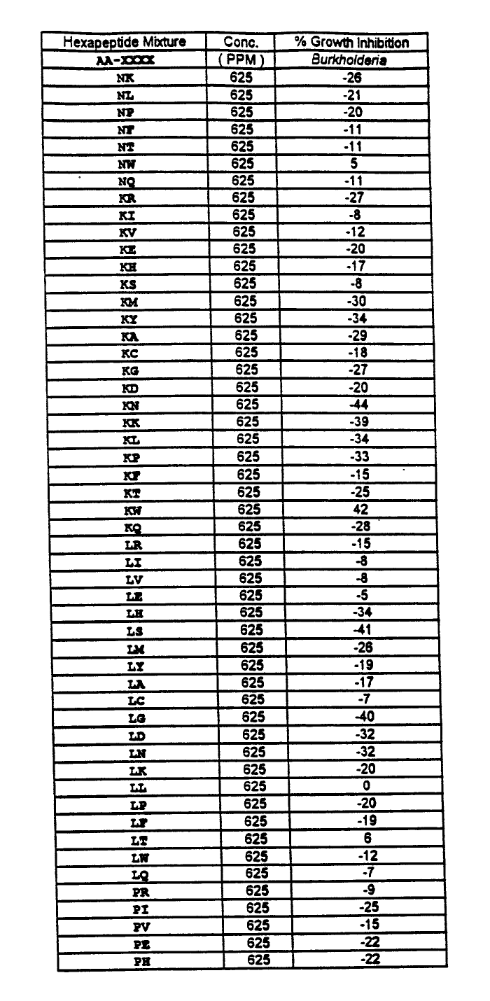

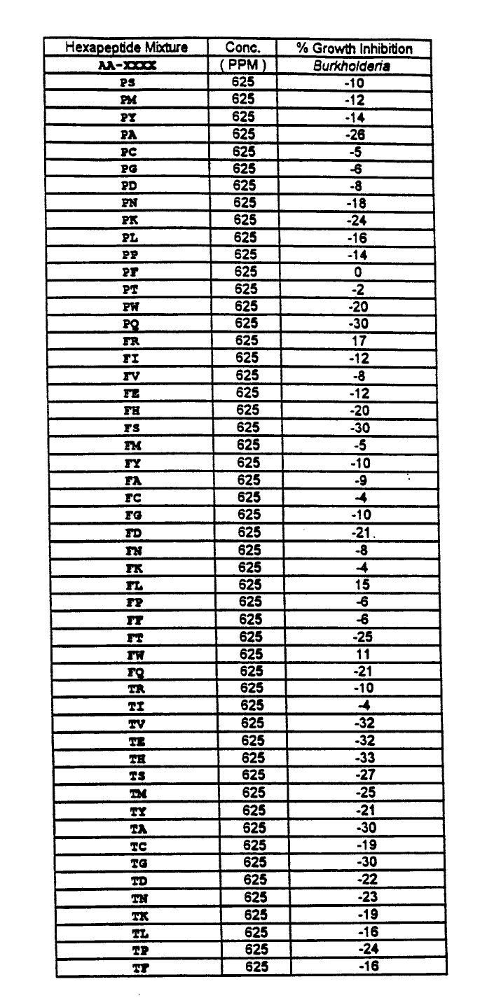

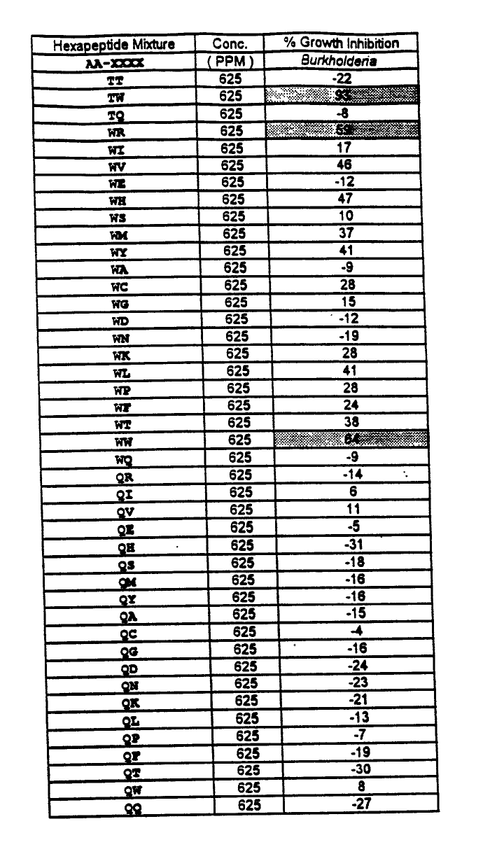

The growth of Burkholderia cepacia ATCC 25416 in the presence of hexapeptide mixtures comprising equimolar concentrations of peptides with defined L-amino acids in positions 1 and 2 and undefined (any of the 20 naturally occurring amino acids) L-amino acids in positions 3, 4, 5 and 6 (the defined amino acids are indicated in column 1 of the attachment) was determined. The peptides were modified to contain an N-terminal acetyl group (at the α-amino group) and a C-terminal NH₂ group. The results of this example are shown in FIG. 1. Peptide mixtures demonstrating a 50% or greater inhibition of growth are highlighted. The peptide mixture consisting of acetyl-TW-X₃X₄X₅X₆-NH₂ exhibited optimal activity.

### Example 3

Antibiofouling compositions for water treatment comprise peptide mixtures of the present invention from about 0.1% to about 50% by weight of the total composition. Other components in the antibiofouling compositions (used at 0.1% to 50%) may include:

2-bromo-2-nitropropane-1,3-diol (BNPD)

β-nitrostyrene (BNS)

dodecylguanidine hydrochloride

2,2-dibromo-3-nitrilopropionamide (DBNPA)

glutaraldehyde

isothiazolin

methylene bis(thiocyanate)

triazines

n-alkyl dimethylbenzylammonium chloride

trisodium phosphate-based antimicrobials

tributyltin oxide

oxazolidines

tetrakis (hydroxymethyl)phosphonium sulfate (THPS)

phenols

chromated copper arsenate

zinc or copper pyrithione

carbamates

sodium or calcium hypochlorite

sodium bromide

halohydantoins (Br, Cl)

Chlorine rates are based on achieving the appropriate concentration of free halogen. Other components in the composition may include biodispersants (about 0.1% to about 15% by weight of the total composition), water, glycols (about 20-30%) or Pluronic (at approximately 7% by weight of the total composition). The concentration of antibiofouling composition for continuous or semi-continuous use is about 5-to about 70 mg/l.

### Example 4

Antibiofouling compositions for industrial water treatment comprise peptide mixtures of the present invention from about 0.1% to about 50% by weight of peptide based on the weight of the total composition. The amount of peptide mixture in antibiofouling compositions for aqueous water treatment may be adjusted depending on the particular peptide mixture and aqueous environment. Shock dose ranges are generally about 20 to about 140 mg/l; the concentration for semi-continuous use is about 0.5× of these concentrations.

### Example 5

Examples of antimicrobial compositions for use as household products include:

| | |
|---|---|
| A. | Powder Automatic Dishwashing Composition |

| | |
|---|---|
| Peptide mixture | 0.00001-50% |
| nonioinic surfactant | 0.4-2.5% |
| sodium metasilicate | 0-20% |
| sodium disilicate | 3-20% |
| sodium triphosphate | 20-40% |
| sodium carbonate | 0-20% |
| sodium perborate | 2-9% |
| tetraacetylethylenediamine | 1-4% |
| sodium sulphate | 5-33% |
| enzymes, including modified enzymes | 0.0001-0.5% |

| | |
|---|---|
| B. | Non-aqueous Liquid Automatic Dishwashing Composition |

| | |
|---|---|
| Peptide mixture | 0.00001-50% |
| liquid nonionic surfactant | 2-10% |
| alkali metal silicate | 3-15% |
| alkali metal phosphate | 20-40% |
| liquid carrier selected from higher | 25-45% |
| glycols, polyglycols, polyoxides, | |
| glycoethers | |
| stabilizer (partial ester of phosphoric | 0.5-7% |
| acid and a C₁₆-C₁₈ alkanol) | |
| foam suppressor (silicone) | 0-1.5% |
| enzymes, including modified enzymes | 0.0001-0.5% |

| | |
|---|---|
| C. | Liquid Automatic Dishwashing Composition |

| | |
|---|---|
| Peptide mixture | 0.00001-50% |
| fatty acid ester sulphonate | 0-30% |
| sodium dodecyl sulphate | 0-20% |
| alkyl polyglycoside | 0-21% |
| oleic acid | 0-10% |
| sodium disilicate monohydrate | 18-33% |
| sodium citrate dihydrate | 18-33% |
| sodium stearate | 0-2.5% |
| sodium perborate monohydrate | 0-13% |
| tetraacetylethylenediamine | 0-8% |
| maleic acid/acrylic acid copolymer | 4-8% |
| enzymes, including modified enzymes | 0.0001-0.5% |

| | |
|---|---|
| D. | Laundry Detergent or Hard Surface Cleaner |

| | |
|---|---|
| Peptide mixture | 0.00001-50% |
| alkyl benzene sulfonic acid | 1-20% |
| sodium C12-15 alkyl sulfate | 0.5-5% |
| ethoxylated C14-15 alkyl sulfate | 0-15% |
| C12 glucose amide | 0-15% |
| ethoxylated C12-15 alcohol | 0-15% |
| fatty acid | 1-15% |
| citric acid | 2-15% |
| C₁₂₋₁₄ alkenyl substituted | 0-15% |
| succinic acid | |
| sodium hydroxide | 0.5-15% |
| ethanol | 1-10% |
| monoethanolamine | 0-10% |
| 1,2-propane diol | 2-10% |
| LipolaseR (100 KLU/g commercial | 0-1% |
| solution) | |

### Example 6

Examples of pharmaceutical compositions for prophylactic or therapeutic treatment include:

| A. | For Vaginal Douches: | |
|---|---|---|
| | Peptide mixture | 0.000001-20% |
| | benzalkonium chloride, parabens or | 0-30% |
| | chlorothymol (other antimicrobial | |
| | agents) | |
| | phenol or menthol (anesthetic or | 10-30% |
| | antipruritics) | |
| | potassium alum (astringent) | 0.4% or 4 g |
| | zinc sulfate (astringent) | 0.4% or 4 g |
| | liquefied phenol | 0.5-5% |
| | glycerin | 10-15% |
| | sodium lauryl sulfate (surface active agent) | 20-50% |
| | sodium borate, sodium bicarbonate or | 10-15% |
| | citric acid (pH altering chemicals) | |
| | pyrogen-free, sterile water | qs to make 1000 ml |
| B. | For Nasal Solutions | |
| | Peptide mixture | 0.000001-10% |
| | chlorobutanol | 0.5-5% |
| | sodium chloride | 0.5-5% |
| | antimicrobial preservatives | 0-70% |
| | pyrogen-free, sterile water | qs to make 100 ml |
| C. | Exilirs | |
| | Peptide mixture | 0.000001-15% |
| | orange oil | 0.1-5% |
| | benzaldehyde | 0.005-5% |
| | sorbitol solution USP | 10-25% |
| | propylene glycol | 40-60% |
| | alcohol | 40-60% |
| | pyrogen-free, sterile water | qs to make 100 ml |
| D. | Otic Solutions | |
| | Peptide mixture | 0.000001-10% |
| | starch glycerin | 10-35% |
| | benzoic acid | 2-10% |
| | glycerin | 70% |
| | pyrogen-free, sterile water | 20% |
| E. | For Inhalations and Inhalants (Solutions) | |
| | Peptide mixture (solubilized) | 0.000001-25% |
| | antioxidants (ex: ascorbic acid) | 0.5-10% |
| | solvent blends (ex: water, ethanol, glycols) | 40-70% |
| | propellants | 5-15% |
| F. | For Inhalations and Inhalants (Suspensions) | |
| | Peptide mixture (micronized & suspended) | 0.000001-25% |
| | dispersing agent (ex: sorbitan trioleate, | 40-50% |
| | oleyl alcohol, oleic acid, lecithin) | |
| | propellants | 5-20% |
| G. | Liniments | |
| | Peptide mixture | 0.000001-20% |
| | ammonium chloride | 10-25% |
| | dilute ammonia solution | 2-20% |
| | oleic acid | 5-25% |
| | turpentine oil | 15-35% |
| | pyrogen-free, sterile water | 50-70% |
| H. | For Water in Oil in Water Emulsion (W/O/W) | |
| | Peptide mixture | 0.000001-20% |
| | isopropyl myristate | 30-60% |
| | sorbitan monooleate | 1-10% |
| | pyrogen-free, sterile water | qs to 100 ml |
| I. | Oil in Water in Oil Emulsion (O/W/O) | |
| | Peptide mixture | 0.000001-20% |
| | soybean oil | 5-20% |
| | ethanol | 10-35% |
| | egg phosphatides | 0.5-10% |
| | Myrj 52 (polyoxyethylene derivative of | 0.1-5% |
| | fatty acids) | |
| | pyrogen-free, sterile water | qs to 100 ml |
| J. | Water in Oil Microemulsion (W/O) | |
| | Peptide mixture | 0.000001-20% |
| | propylene glycol esters of capric/ | 5-50% |
| | caprylic acids | |
| | polyoxyethylene (50) sorbitan esters | 8-20% |
| | polyoxyethyleneglycerol triricinoleate | 8-20% |
| | propylene glycol | 20-30% |
| K. | Gels | |
| | Peptide mixture | 0.00001-20% |
| | sodium alginate (gelling agent) | 2-10% |
| | glycerin | 2-10% |
| | methyl hydroxybenzoate | 0.1-5% |
| | pyrogen-free, sterile water | qs to 100 ml |
| L. | Creme-Lotions | |
| | Peptide mixture | 0.01-15% |
| | anhydrous lanolin | 15-40% |
| | mineral oil | 5-35% |
| | olive oil | 5-35% |
| | ethyl alcohol | 5-35% |
| | pyrogen-free, sterile water | 5-20% |
| | glycerin | 5-20% |
| | Tween 80 | 0.5-5% |
| | Polyvinylpyrrolidone (PVP) | 0.5-5% |
| | sodium dodecyl sulfate | 0.1-5% |
| M. | Oleaginous Base Topical Formulations | |
| | Peptide mixture | 0.01-5% |
| | anhydrous lanolin | 10-40% |
| | mineral oil | 10-40% |
| | olive oil | 10-40% |
| | Tween 80 | 5-20% |
| N. | Oleaginous Base Ointments | |
| | Peptide mixture | 0.01-10% |
| | anhydrous lanolin | 10-45% |
| | white petrolatum | 10-45% |
| | olive oil | 10-45% |
| | Tween 80 | 5-35% |
| O. | Intravenous Admixtures | |
| | Peptide mixture | 0.000001-10% |
| | polyoxyethylene glycol monoester of saturated | 5-75% |
| | hydroxylated fatty acid | |
| | polyethylene glycol | 2-50 ml |
| | 96% ethanol | qs 100 ml |
| | solution diluted with isotonic saline, glucose, | |
| | dextran, fructose or mannitol solution. | |
| P. | Other Parenteral Admixtures | |
| | Peptide mixture | 0.0001-10% |
| | soybean oil | 5-35% |
| | acetylated monoglycerides | 1-25% |
| | egg yolk phosphatides | 0.1-10% |
| | glycerol | 0.1-10% |
| | pyrogen-free, sterile water | qs 100 ml |
| Q. | Opthalmic Solutions | |
| | Peptide mixture | 0.000001-10% |
| | sodium chloride USP | 0.5-10% |
| | benzalkonium chloride | 1:10,000 |
| | pyrogen-free, sterile water | qs 100 ml |
| R. | Topical ointments | |
| | Peptide mixture | 0.00001-20% |
| | methylparaben | 0.1-10 g |
| | propylparaben | 0.1-10 g |
| | sodium lauryl sulfate | 5-25% |
| | propylene glycol | 5-25% |
| | stearyl alcohol | 10-45% |
| | white petrolatum | 10-45% |
| | pyrogen-free, sterile water | 20-60% |
| S. | Emulsion type topical solutions | |
| | Peptide mixture | 0.0001-20% |
| | transcutol | 5-45% |
| | polyoxyethylene glycolated hydrogenated | 1-15% |
| | castor oil | |
| | transesterified triglyceride (Labrafil) | 5-35% |
| | glycerol monostearate | 5-40% |
| | white petrolatum | 20-60% |
| T. | Space Spray | |
| | Peptide mixture | 2-20% |
| | propellant | 80-98% |
| U. | Surface-coating Spray | |
| | Peptide mixture | 1-75% |
| | propellant | 25-99% |
| V. | Foam Spray (edible) | |
| | Peptide mixture | up to 50% |
| | vegetable oil (ex: peanut, cottonseed, soybean) | 40-90% |
| | emulsifier (ex: glyceryl monostearate) | 1-10% |
| | propellant (ex: propane) | 1-10% |
| W. | Other foam Spray | |
| | Peptide mixture | up to 50% |
| | ethanol | 46-66% |
| | surfactant (ex: nonionic, anionic or cationic) | 0.5-5% |
| | pyrogen-free, sterile water | 28-42% |
| | propellant (ex: propane) | 3-15% |
| X. | Soft gelatin capsules | |
| | Peptide mixture | 0.0001-15% |
| | caprylic acid | 2-25% |
| | capric acid | 2-25% |
| | lauric acid | 5-50% |
| | myristic acid | 2-25% |
| | palmitic acid | 5-15% |
| | stearic acid | 5-15% |
| | monoacylglyceride | 5-50% |
| | diacylglyceride | 5-40% |
| | triacylglyceride | 5-60% |
| | silicon dioxide | 0.05-3% |
| Y. | Hard gelatin capsules | |
| | Peptide mixture | 0.001-60% |
| | stearate 1500 | 15-30% |
| | Eudragit S 100 | 25-69% |

### Example 7

Examples of doses of pharmaceutical compositions comprising peptides of the present invention include:

| | | | |
|---|---|---|---|
| A. | Nebulizer | 5 to 200 | mg/ml |
| B. | Metered dose inhaler | 0.5 to 45 | mg |
| C. | Dry powder inhaler | 0.5 to 45 | mg |
| D. | Intramuscular, intravenous | 1 to 10 | mg/kg |
| | or intraperitoneal injection | | |

### Example 8

Examples of diseases or infections treatable by pharmaceutical compositions comprising peptide mixtures of the present invention include:

| DISEASES/INFECTIONS | DOSE |
|---|---|
| Cystic fibrosis | 0.5-45 mg (inhaler) |
| Bronchitis | 0.01-100 mg/kg (oral) |
| Burn or wound infections | 0.000001-20% (cream) |
| Otitis media | 0.000001-20% (ear drops) |
| Urinary tract infection | 0.01-100 mg/kg (oral) |
| Sinusitis | 0.01-100 mg/kg (oral) |
| Periodontitis | 0.0001-1% (mouth rinse) |

### Example 9

Examples of hygiene compositions for personal care use comprising peptide mixtures of the present invention include:

| A. | Facial Cleanser | |
|---|---|---|
| | Peptide mixture | 0.0001-20% |
| | ammonium laureth sulfate | 28-32% |
| | disodium EDTA | 0.01-0.1% |
| | cocamidopropyl betaine | 6-9% |
| | cocamidopropyl phosphatidyl PG- | 1-3% |
| | dimonium chloride | |
| | cocamide DEA | 1-3% |
| | lactic acid | 0-3% |
| | glycerin | 1-5% |
| | propylene glycol, imidazolidinyl | 0.5-1% |
| | urea, methylparaben, propylparaben | |
| | pyrogen-free, sterile deionized water | 50-55% |
| | sodium hydroxide | 0.5-10% |
| B. | Cream | |
| | Peptide mixture | 0.00001-15% |
| | behentrimonium methosulfate, | 0.5-4% |
| | cetearyl alcohol | |
| | Miglyol 840 | 5-10% |
| | Arlacel 165 | 5-12% |
| | phenyl trimethicone | 0.5-4% |
| | glycerin | 0.5-6% |
| | propylene glycol, diazolidinyl | 0.5-2% |
| | urea, methylparaben, propylparaben | |
| | xanthan gum | 0.05-2% |
| | magnesium aluminum silicate | 0.05-5% |
| | silica | 0.05-3% |
| | Tween 60 | 0.05-2% |
| | lactic acid | 1-20% |
| | sodium hydroxide | 0.5-12% |
| | cyclomethicone | 0.5-2% |
| | pyrogen-free, sterile deionized water | 30-70% |
| C. | Cream | |
| | Peptide mixture | 0.00001-15% |
| | cetostearyl alcohol | 0.3-15% |
| | hydrogenated lanolin | 0.5-15% |
| | ethyl p-hydroxybenzoate | 0.03-5% |
| | polyoxyethylene (20) sorbitan | 0.2-10% |
| | monopalmitate | |
| | glycerol monostearate | 0.2-10% |
| | sodium N-stearoylglutamate | 0.05-5% |
| | retinol acetate | 0.2-10% |
| | perfume | 0.003-5% |
| | 1,3-butylene glycol | 0.5-15% |
| | polyethylene glycol 1500 | 0.5-15% |
| | pyrogen-free, sterile deionized water | balance |
| D. | Sun-screening Cream | |
| | Peptide mixture | 0.000001-15% |
| | decamethylcyclopentasiloxane | 3-50% |
| | liquid paraffine | 0.5-15% |
| | polyoxyalkylene-modified | 0.1-5% |
| | organopolysiloxane | |
| | distearyldimethylammonium chloride | 0.06-5% |
| | perfume | 0.03-5% |
| | titanium oxide | 1-25% |
| | zinc oxide | 0.5-15% |
| | talc | 0.2-15% |
| | glycerin | 0.5-20% |
| | magnesium aluminum silicate | 0.1-10% |
| | pyrogen-free, sterile deionized water | balance |
| E. | Lotion | |
| | Peptide mixture | 0.00001-20% |
| | magnesium aluminum silicate | 0.2-0.5% |
| | xanthan gum | 0.1-0.3% |
| | glyceryl stearate, PEG-100 stearate | 5-10% |
| | Tween 60 | 0.5-2% |
| | ceteareth alcohol | 0.5-2% |
| | propylene glycol, diazolidinyl urea, | 0.5-2% |
| | methylparaben, propylparaben | |
| | glycerin | 2-6% |
| | Miglyol 840 | 8-12% |
| | phenyl trimethicone | 1-3% |
| | cyclomethicone | 0.5-2% |
| | lactic acid | 1-20% |
| | sodium hydroxide | 0.5-13% |
| | pyrogen-free, sterile deionized water | 35-38% |
| F. | Clear Lotion | |
| | Peptide mixture | 0.00001-15% |
| | tocopherol acetate | 0.001-5% |
| | glycerin | 0.4-10% |
| | 1,3-butylene glycol | 0.4-10 |
| | ethanol | 0.8-15% |
| | polyoxyethylene (60) hardened castor oil | 0.05-5% |
| | methyl p-hydroxybenzoate | 0.02-5% |
| | citric acid | 0.005-5% |
| | sodium citrate | 0.01-5% |
| | perfume | 0.005-5% |
| | pyrogen-free, sterile deionized water | balance |
| G. | Milky Lotion | |
| | Peptide mixture | 0.00001-15% |
| | stearic acid | 0.15-5% |
| | cetyl alcohol | 0.05-5% |
| | polyoxyethylene (10) monooleate | 0.2-10% |
| | L-arginine | 0.03-6% |
| | sodium L-glutamate | 0.002-5% |
| | PCA-NA | 0.005-5% |
| | 2-aminoethylthiosulfonic acid | 0.02-5% |
| | 2-aminoethylsulfinic acid | 0.001-5% |
| | propylene glycol | 0.5-10% |
| | glycerin | 0.3-10% |
| | ethanol | 0.3-10% |
| | ethyl p-hydroxybenzoate | 0.03-3% |
| | perfume | 0.003-3% |
| | carboxyvinyl polymer | 0.01-5% |
| | pyrogen-free, sterile deionized water | balance |
| H. | Sun-screening Milky Lotion | |
| | Peptide mixture | 0.00001-15% |
| | stearic acid | 0.2-5% |
| | cetyl alcohol | 0.05-5% |
| | liquid paraffin | 1-20% |
| | polyoxyethylene (10) oleate | 0.1-5% |
| | sorbitan trioleate | 0.1-5% |
| | perfume | 0.02-2% |
| | 1,3-butylene glycol | 0.5-5% |
| | dipropylene glycol | 0.3-3% |
| | carboxyvinyl polymer | 0.01-5% |
| | trisodium edetate | 0.005-3% |
| | triethanolamine | 0.04-5% |
| | silica | 0.2-2% |
| | talc | 0.2-2% |
| | titanium oxide | 0.3-3% |
| | zinc oxide | 0.3-3% |
| | pyrogen-free, sterile deionized water | balance |
| I | Hair Conditioner | |
| | Peptide mixture | 0.001-20% |
| | pyrogen-free, sterile deionized water | 89-92% |
| | dimethyl hydroxymethyl pyrazole | 0.5-5% |
| | panthenol | 0.1-0.3% |
| | disodium EDTA | 0.02-.1% |
| | cetearyl alcohol, ceteareth-20 | 1-2% |
| | stearyl alcohol | 4-6% |
| | cetrimonium bromide | 4-6% |
| | jojoba oil | 0.2-0.5% |
| | acetamide MEA | 0.5-2% |
| | lactamide MEA | 0.5-2% |
| J. | Hair Shampoo | |
| | Peptide mixture | 0.001-20% |
| | anionic surfactant | 5-15% |
| | (polyoxyethylenealkyl sulfate) | |
| | cationic surfactant | 0.5-2.5% |
| | (distearyl dimethylammonium chloride) | |
| | amphoteric surfactant | 5-15% |
| | (alkylamine oxide) | |
| | thickener | 0.5-15% |
| | (isostearic acid diethanolamide) | |
| | wetting agent (propylene glycol) | 1-20% |
| | lower alcohol (ethanol) | 1-15% |
| | perfume | proper amount |
| | pyrogen-free, sterile deionized water | balance |
| K. | Antiperspirant/Deodorant Solution | |
| | Peptide mixture | 0.0001-20% |
| | aluminum chlorohydrate | 10-40% |
| | SD alcohol 40 | 25-35% |
| | Transcutol ethoxydiglycol | 5-10% |
| | Tween 20 | 0.5-1% |
| | cocamidopropyl phosphatidyl PG-dimonium | 1-2% |
| | chloride | |
| | pyrogen-free, sterile deionized water | 20-25% |
| L. | Mouthwash | |
| | Peptide mixture | 0.001-20% |
| | SD alcohol | 4-35% |
| | selenomethionine | 0.2-5% |
| | calcium gluconate | 0.25-5% |
| | L-glutathione | 0.10-4% |
| | xylitol-sweetener | 1-10% |
| | coloring agents | 0.1-3% |
| | flavoring agents | 0.1-5% |
| | pyrogen-free, sterile deionized water | balance |
| M. | Toothpaste | |
| | Peptide mixture | 0.00001-10% |
| | glycerol | 2-50% |
| | magnesium carbonate | 0.35-10% |
| | sodium fluoride | 0.35-10% |
| | zinc acetate | 0.05-10% |
| | L-glutathione | 0.01-5% |
| | L-selenomethionine | 0.005-5% |
| | ascorbic acid | 0.15-5% |
| | N-acetylcysteine | 0.01-10% |
| | benzalkonium chloride | 0.01-10% |
| | polyvinyl pyrrolidone | 0.75-10% |
| | xylitol (sweetner) | 0.025-5% |
| | coloring agent | 0.02-3% |
| | peppermint (flavor) | 0.02-3% |
| | pyrogen-free, sterile deionized water | balance |
| N. | Tooth gels | |
| | Peptide mixture | 0.00001-10% |
| | glycerin | 2-50% |
| | poloxamer | 10-25% |
| | ascorbic acid | 0.15-5% |
| | sodium lauryl sulfate | 0.12-12% |
| | peppermint oil | 0.1-5% |
| | alpha tocopherol | 0.075-8% |
| | calcium laurate | 0.025-5% |
| | selenomethionine | 0.02-5% |
| | sodium fluoride | 0.02-5% |
| | L-glutathione | 0.01-10% |
| | coloring agent | 0.01-5% |
| | xylitol (sweetner) | 0.15-20% |
| | zinc acetate | 0.015-3% |
| | pyrogen-free, sterile deionized water | balance |
| O. | Body Washes | |
| | Peptide mixture | 0.001-20% |
| | dimethylsiloxane-methyl siloxane | 0.5-2.5% |
| | copolymer | |
| | potassium cocoyl hydrolyzed | 5-40% |
| | collagen | |
| | coconut oil potassium soap (40%) | 0.5-15% |
| | coconut oil fatty acid | 1-15% |
| | diethanolamide | |
| | lauric acid diethanolamide | 1-15% |
| | p-hydroxybenzoates and | 0.05-2.5% |
| | phenoxyethanol | |
| | pyrogen-free, sterile deionized water | balance |
| P. | Ointment | |
| | Peptide mixture | 0.00001-20% |
| | tocopherol acetate | 0.05-5% |
| | retinol palmitate | 0.1-10% |
| | stearyl alcohol | 1-30% |
| | Japan wax | 2-40% |
| | polyoxyethylene (10) monooleate | 0.025-5% |
| | glycerol monostearate | 0.03-10% |
| | vaseline | 5-45% |
| | pyrogen-free, sterile deionized water | balance |

### Example 10

Examples of peptide compositions for medical devices include:

| | |
|---|---|
| | Polyurethane Adhesive Film Containing Pharmaceutical |
| A. | Composition |

| | |
|---|---|
| Peptide mixture | 0.025-20% |
| polyoxyethylene glycol | 2-5% |
| polyurethane adhesive solution | 10-25% |

when coated and dried results in a tacky, adhesive film for dressing wounds

| | |
|---|---|
| B. | Suture Containing Pharmaceutical Composition |

| | |
|---|---|
| Peptide mixture | 0.025-20% |
| polyoxyethylene glycol | 2-5% |

suture is dipped in solution above and excess is wiped away with a paper towel for dressing wounds

| | |
|---|---|
| C. | Catheter Containing Pharmaceutical Composition |

| | |
|---|---|
| Peptide mixture | 0.025-20% |
| polyoxyethylene glycol | 2-5% |

solution above is applied onto the surface of polyurethane catheter

| | |
|---|---|
| | Foam Dressing Containing Pharmaceutical |
| D. | Composition |

| | |
|---|---|
| Peptide mixture | 0.025-20% |
| polyoxyethylene glycol | 2-5% |

3.5 g of above solution is mixed with 5.5 g polyurethane prepolymer and then 5.5 g water to form a foam which is dried and then sliced to produce foam dressings

| | |
|---|---|
| | Hydrocolloid Dressing Containing Pharmaceutical |
| E. | Composition |

| | |
|---|---|
| Peptide mixture | 0.025-20% |
| polyoxyethylene glycol | 2-5% |

2 g of above solution is mixed with 4 g sodium carboxymethyl cellulose and then 4 g polyurethane prepolymer. Mixture is pressed between a polyurethane film and silicone-treated polyester liner to make a 2.5 mm thick treated hydrocolloid matrix which is allowed to cure for 24 hours.

### Example 11

Peptide Compositions For Textiles

Peptide mixtures of the present invention can be applied by coating or spinning effective amounts of peptide onto or into the desired polymer. The peptides can be prepared in an aqueous solution to use as a coating solution or with a polymer. The coating solutions can contain small water-soluble molecules that do not interfere with the antimicrobial action of the peptide. A peptide and polymer solution or mixture can be made and undergo casting or formation to the desired shaped article, fiber or film. The shaped article, fiber or film can then be put in water or methanol, and air dry or dry under an appropriate atmosphere to prevent oxidative reactions.

| | |
|---|---|
| Peptide mixture | 0.01-15% |
| Polymer solution | 10%-15% |
| (e.g., containing wool or cotton) | |

The resulting solution can be put into a microscale spinning apparatus and fiber is formed while wet with methanol. The antimicrobial activity of the peptides can be tested in tubes containing LB media innoculated with the peptide-containing fiber and E.coli growing at log phase (1×10⁶ to 1×10⁷ cells/ml). Aliquots can be taken from the culture tube at periodic intervals for absorbance readings at 600 nm (uv/vis) in a microcuvette.

### Example 12

Example of peptide compositions comprising liposomes:

Composition comprising liposomes and acetyl-TWX₃X₄X₅X₆-NH₂ for inhibition of microbial growth in cell culture at 37° C.

| | | |
|---|---|---|
| Peptide mixture | 0.5-50 | μg |
| Liposome (unilamellar or | 2-400 | μg |
| (multilamellar) | | |

Viable cell counts can be performed after 3 hours to show greater than 90% reduction in growth of B. cepacia in comparison to control cultures.

### Example 13

Antiviral Susceptibility Testing

The antiviral activity of acetyl-TWX₃X₄X₅X₆-NH₂ may be determined. The peptide is first evaluated for cytotoxicity. Vero cells (ATCC CCL81) are grown to confluency in 96-well microtiter plates in Eagles Minimal Essential Medium (E-MEM) supplemented with 10% fetal bovine serum (FBS), 100 units/ml penicillin, 2.5 μg/ml Amphotericin B and 10 μg/ml gentamicin (total volume 0.2 ml). Plates are incubated at 37° C. in a humidified atmosphere of 6% CO₂. Spent culture medium is removed and each well receives 0.2 ml of the appropriate peptide dilution or cell culture medium (cell control wells). The plates are incubated at 37° C., 6% CO₂ for 4-8 days, after which the cells are examined microscopically and a microtetrazolium assay is performed using 2,3-bis[(phenylamino) carbonyl]-2H-tetrazolium hydroxide (XTT).

The peptide mixture is evaluated for antiviral activity using Herpes Simplex Virus Type 1 in a plaque reduction assay. Microtiter plates (24 well) are seeded with Vero cells to confluency. The supernatant medium is removed by aspiration and each well receives 0.5 ml E-MEM with 5% FBS. Virus (0.2 ml) is added to the medium in the test and control wells to achieve 50 plaque-forming units (pfu) per well. After virus attachment the inoculum is removed and replaced with 1 ml medium containing the appropriate dilution of peptide. Plates are incubated at 37° C. under 6% CO₂ until plaques are sufficiently well defined to count (2-5 days). The cells are fixed with formalin (10%) in phosphate buffered saline and stained with crystal violet. Plaques are then counted and the EC₅₀ (peptide concentration that produces a 50% reduction in plaque formation) is calculated.

### Example 14

Antiparasitic Susceptibility Tesing

Methods for antiparasitic susceptibility testing are described in pages 1653-1662 of ANTIPARASITIC AGENTS AND SUSCEPTIBILITY TESTS, Nguyen-Dinh, P., Secor, W. E., and MANUAL OF CLINICAL MICROBIOLOGY (7th Edition), Murray, P. R., Baron, E. J., Pfaller, M. A., Tenover, F. C., Yolken, R. H. (eds.), American Society for Microbiology Press, Washington, DC, 1999.

Testing for Plasmodium falciparum

P. falciparum is added as parasite-infected red blood cells (at concentrations ranging from 0.05 to 0.5%) to flasks containing 50 ml human red blood cells in RPMI 1640 medium plus [³H]-labeled hypoxanthine (10 μM; 50 μCi) for 150 ml final volume. The red blood cells are incubated for 1 week at 37° C. under 5% CO₂. Test peptide (e.g., acetyl-TWX₃X₄X₅X₆-NH₂) is then added at final concentrations of 0 to 500 μg/ml and the mixtures are incubated an additional 24 hr. The cells undergo filtration and hypoxanthine uptake is measured by liquid scintillation counting to determine P. falciparum viability.

### Example 15

The hemolytic activity of sample peptides can be determined using human erythrocytes. Assays are performed in 96-well flat bottom microtiter plates in a total volume of 100 μl. The assay components (final concentration) are 0.25% human red blood cells (RBCs) and peptide mixture at concentrations of 0 to 500 μg/ml. Plates incubate for 1 hr at 37° C. and then undergo centrifugation at 2800 rpm for 5 min. The supernatant is separated from the pellet and the optical density of the supernatant at 414 nm is measured. The concentration of peptide mixture to lyse 50% of the RBCs is the hemolytic dose (HD) or HD₅₀.

Although the invention has been described with reference to particular means, materials and embodiments, it is to be understood that the invention is not limited to the particulars disclosed, and extends to all equivalents within the scope of the claims.

We claim:

1. An antimicrobial composition comprising a plurality of hexapeptides
wherein for each hexapeptide, the amino acid in the first position, based on numbered amino acids from N-terminus to C-terminus, is selected from the group consisting of arginine, lysine, methionine, serine, threonine and tryptophan;
the amino acid in the second position, based on numbered amino acids from N-terminus to C-terminus, is selected from the group consisting of arginine, histidine, cysteine, threonine, tyrosine, and tryptophan;
the amino acids in positions three through six, based on numbered amino acids from N-terminus to C-terminus, are any amino acid; and
wherein the first two amino acids of said hexapeptides are other than arginine-arginine, tryptophan-tryptophan, tryptophan-cysteine, tryptophan-lysine, arginine-tryptophan, or threonine-arginine.

2. The antimicrobial composition of claim 1 wherein the amino acids in the first and second positions of said peptides, based on numbered amino acids from N-terminus to C-terminus, are selected from the group consisting of Arg-Tyr, Arg-Cys, Ser-Thr, Met-Trp, Lys-Trp, Thr-Trp, Trp-Arg, Trp-His, and Trp-Tyr.

3. The antimicrobial composition of claim 1 wherein said peptides are incorporated into a polymer.

4. The antimicrobial composition of claim 3 wherein said polymer is selected from the group consisting of a polysaccharide, a glycol polymer, a polyester, a polyurethane, a polyacrylate, a polyacrylonitrile, a polyamide, a polyolefin, a polystyrene, a vinyl polymer, a polypropylene, silk, a biopolymer, and mixtures thereof.

5. An antimicrobial composition comprising a plurality of peptides, wherein said peptides each are represented by Formula I: 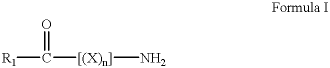
wherein:
X represents any amino acid except glutamate or aspartate;
n=6;
R₁ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₅-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₃ is independently hydrogen; C₁-C₄ alkyl; or phenyl optionally substituted with at least one R₈;
R₄ is independently hydrogen; C₁-C₈ alkyl; or phenyl optionally substituted with at least one R₈;
R₅ is independently C₁-C₆ alkyl; C₁-C₆ alkoxy; C₁-C₆ haloalkyl; halogen; C₂-C₈ alkynyl; C₁-C₆ thioalkyl; phenyl or phenoxy each optionally substituted with at least one R₈; cyano; nitro; C₁-C₆ haloalkoxy; C₁-C₆ haloalkythio; C₂-C₆ alkenyl; C₂-C₆ haloalkenyl; acetyl; CO₂CH₃; or N(C₁-C₂ alkyl)₂;
R₆ is independently methyl; ethyl; methoxy; methylthio; halogen; or trifluoromethyl;
R₇ is independently halogen; and
R₈ is independently halogen; C₁-C₄ alkyl; C₁-C₄ alkoxy; C₁-C₄ haloalkyl; nitro; or cyano;
wherein:
the amino acid in the first position, based on numbered amino acids from N-terminus to C-terminus, is selected from the group consisting of arginine, lysine, methionine, serine, threonine and tryptophan;
the amino acid in the second position, based on numbered amino acids from N-terminus to C-terminus, is selected from the group consisting of arginine, histidine, cysteine, threonine, tyrosine, and tryptophan; and
the amino acids in positions three through six, based on numbered amino acids from N-terminus to C-terminus, are any amino acid;
wherein the first two amino acids of said hexapeptides are other than arginine arginine, tryptophan-tryptophan, tryptophan-cysteine, tryptophan-lysine, arginine-tryptophan, or threonine-arginine.

6. The antimicrobial composition of claim 5 wherein the amino acids in the first and second positions of said peptides, based on numbered amino acids from N-terminus to C-terminus, are selected from the group consisting of Arg-Tyr, Arg-Cys, Ser-Thr, Met-Trp, Lys-Trp, Thr-Trp, Trp-Arg, Trp-His, and Trp-Tyr.

7. The antimicrobial composition of claim 5 wherein said peptides are incorporated into a polymer.

8. The antimicrobial composition of claim 7 wherein said polymer is selected from the group consisting of a polysaccharide, a glycol polymer, a polyester, a polyurethane, a polyacrylate, a polyacrylonitrile, a polyamide, a polyolefin, a polystyrene, a vinyl polymer, a polypropylene, silk, a biopolymer, and mixtures thereof.

9. An antimicrobial composition comprising a plurality of peptides, wherein said peptides each are represented by Formula II: 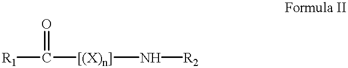
wherein:
X represents any amino acid except glutamate or aspartate;
n=6;
R₁ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₅-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₂ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₁-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₃ is independently hydrogen; C₁-C₄ alkyl; or phenyl optionally substituted with at least one R₈;
R₄ is independently hydrogen; C₁-C₈ alkyl; or phenyl optionally substituted with at least one R₈;
R₅ is independently C₁-C₆ alkyl; C₁-C₆ alkoxy; C₁-C₆ haloalkyl; halogen; C₂-C₈ alkynyl; C₁-C₆ thioalkyl; phenyl or phenoxy each optionally substituted with at least one R₈; cyano; nitro; C₁-C₆ haloalkoxy; C₁-C₆ haloalkythio; C₂-C₆ alkenyl; C₂-C₆ haloalkenyl; acetyl; CO₂CH₃; or N(C₁-C₂ alkyl)₂;
R₆ is independently methyl; ethyl; methoxy; methylthio; halogen; or trifluoromethyl;
R₇ is independently halogen; and
R₈ is independently halogen; C₁-C₄ alkyl; C₁-C₄ alkoxy; C₁-C₄ haloalkyl; nitro; or cyano;
wherein:
the amino acid in the first position, based on numbered amino acids from N-terminus to C-terminus, is selected from the group consisting of arginine, lysine, methionine, serine, threonine and tryptophan;
the amino acid in the second position, based on numbered amino acids from N-terminus to C-terminus, is selected from the group consisting of arginine, histidine, cysteine, threonine, tyrosine, and tryptophan; and
the amino acids in positions three through six, based on numbered amino acids from N-terminus to C-terminus, are any amino acid.

10. The antimicrobial composition of claim 9 wherein the amino acids in the first and second positions of said peptide, based on numbered amino acids from N-terminus to C-terminus, are selected from the group consisting of Arg-Tyr, Arg-Cys, Ser-Thr, Met-Trp, Lys-Trp, Thr-Trp, Trp-Arg, Trp-His, and Trp-Tyr.

11. The antimicrobial composition of claim 9 wherein said peptides are incorporated into a polymer.

12. The antimicrobial composition of claim 11 wherein said polymer is selected from the group consisting of a polysaccharide, a glycol polymer, a polyester, a polyurethane, a polyacrylate, a polyacrylonitrile, a polyamide, a polyolefin, a polystyrene, a vinyl polymer, a polypropylene, silk, a biopolymer, and mixtures thereof.

13. An antimicrobial composition comprising a plurality of hexapeptides and at least one carrier, wherein for each hexapeptide:
the amino acid in the first position, based on numbered amino acids from N-terminus to C-terminus, is selected from the group consisting of arginine, lysine, methionine, serine, threonine and tryptophan;
the amino acid in the second position, based on numbered amino acids from N-terminus to C-terminus, is selected from the group consisting of arginine, histidine, cysteine, threonine, tyrosine, and tryptophan;
the amino acids in positions three through six, based on numbered amino acids from N-terminus to C-terminus, are any amino acid; and
wherein the first two amino acids of said hexapeptides are other than arginine-arginine, tryptophan-tryptophan, tryptophan-cysteine, tryptophan-lysine, arginine-tryptophan, or threonine-arginine.

14. The antimicrobial composition of claim 13 wherein the amino acids in the first and second positions of said peptides, based on numbered amino acids from N-terminus to C-terminus, are selected from the group consisting of Arg-Tyr, Arg-Cys, Ser-Thr, Met-Trp, Lys-Trp, Thr-Trp, Trp-Arg, Trp-His, and Trp-Tyr.

15. The antimicrobial composition of claim 13 wherein said carrier is selected from the group consisting of a pharmaceutically acceptable carrier, an industrially acceptable carrier, a household product, and a personal care composition.

16. An antimicrobial composition comprising a plurality of hexapeptides and at least one carrier, wherein said each hexapeptide is represented by Formula I: 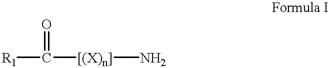
wherein:
X represents any amino acid except glutamate or aspartate;
n=6;
R₁ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₁-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₃ is independently hydrogen; C₁-C₄ alkyl; or phenyl optionally substituted with at least one R₈;
R₄ is independently hydrogen; C₁-C₈ alkyl; or phenyl optionally substituted with at least one R₈;
R₅ is independently C₁-C₆ alkyl; C₁-C₆ alkoxy; C₁-C₆ haloalkyl; halogen; C₂-C₈ alkynyl; C₁-C₆ thioalkyl; phenyl or phenoxy each optionally substituted with at least one R₈; cyano; nitro; C₁-C₆ haloalkoxy; C₁-C₆ haloalkythio; C₂-C₆ alkenyl; C₂-C₆ haloalkenyl; acetyl; CO₂CH₃; or N(C₁-C₂ alkyl)₂;
R₆ is independently methyl; ethyl; methoxy; methylthio; halogen; or trifluoromethyl; and
R₇ is independently halogen;
R₈ is independently halogen; C₁-C₄ alkyl; C₁-C₄ alkoxy; C₁-C₄ haloalkyl; nitro; or cyano;
wherein:
the amino acid in the first position, based on numbered amino acids from N-terminus to C-terminus, is selected from the group consisting of arginine, lysine, methionine, serine, threonine and tryptophan;
the amino acid in the second position, based on numbered amino acids from N-terminus to C-terminus, is selected from the group consisting of arginine, histidine, cysteine, threonine, tyrosine, and tryptophan; and
the amino acids in positions three through six, based on numbered amino acids from N-terminus to C-terminus, are any amino acid;
wherein the first two amino acids of said hexapeptides are other than arginine-arginine, tryptophan-tryptophan, tryptophan-cysteine, tryptophan-lysine, arginine-tryptophan, or threonine-arginine.

17. The antimicrobial composition of claim 16 wherein the amino acids in the first and second positions of said peptides, based on numbered amino acids from N-terminus to C-terminus, are selected from the group consisting of Arg-Tyr, Arg-Cys, Ser-Thr, Met-Trp, Lys-Trp, Thr-Trp, Trp-Arg, Trp-His, and Trp-Tyr.

18. The antimicrobial composition of claim 16 wherein said carrier is selected from the group consisting of a pharmaceutically acceptable carrier, an industrially acceptable carrier, a household product, and a personal care composition.

19. An antimicrobial composition comprising a plurality of hexapeptides and at least one carrier, wherein said each hexapeptide is represented by Formula II: 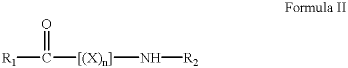
wherein:
X represents any amino acid except glutamate or aspartate;
n=6;
R₁ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₅-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₂ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₅-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₃ is independently hydrogen; C₁-C₄ alkyl; or phenyl optionally substituted with at least one R₈;
R₄ is independently hydrogen; C₁-C₈ alkyl; or phenyl optionally substituted with at least one R₈;
R₅ is independently C₁-C₆ alkyl; C₁-C₆ alkoxy; C₁-C₆ haloalkyl; halogen; C₂-C₈ alkynyl; C₁-C₆ thioalkyl; phenyl or phenoxy each optionally substituted with at least one R₈; cyano; nitro; C₁-C₆ haloalkoxy; C₁-C₆ haloalkythio; C₂-C₆ alkenyl; C₂-C₆ haloalkenyl; acetyl; CO₂CH₃; or N(C₁-C₂ alkyl)₂;
R₆ is independently methyl; ethyl; methoxy; methylthio; halogen; or trifluoromethyl;
R₇ is independently halogen; and
R₈ is independently halogen; C₁-C₄ alkyl; C₁-C₄ alkoxy; C₁-C₄ haloalkyl; nitro; or cyano.

20. The antimicrobial composition of claim 19 wherein the amino acids in the first and second positions, based on numbered amino acids from N-terminus to C-terminus, are selected from the group consisting of Arg-Tyr, Arg-Cys, Ser-Thr, Met-Trp, Lys-Trp, Thr-Trp, Trp-Arg, Trp-His, and Trp-Tyr.

21. The antimicrobial composition of claim 19 wherein said carrier is selected from the group consisting of a pharmaceutically acceptable carrier, an industrially acceptable carrier, a household product, and a personal care composition.

22. A method for preventing, inhibiting, or terminating the growth of at least one microbe comprising administering an antimicrobial amount of a plurality of hexapeptides and at least one carrier, wherein for each hexapeptide:
the amino acid in the first position, based on numbered amino acids from N-terminus to C-terminus, is selected from the group consisting of arginine, lysine, methionine, serine, threonine and tryptophan;
the amino acid in the second position, based on numbered amino acids from N-terminus to C-terminus, is selected from the group consisting of arginine, histidine, cysteine, threonine, tyrosine, and tryptophan;
the amino acids in positions three through six, based on numbered amino acids from N-terminus to C-terminus, are any amino acid; and
wherein the first two amino acids of said hexapeptides are other than arginine-arginine, tryptophan-tryptophan, tryptophan-cysteine, tryptophan-lysine, arginine-tryptophan, or threonine-arginine.

23. The method of claim 22 wherein said microbe comprises Burkholderia cepacia.

24. A method for preventing, inhibiting, or terminating the growth of at least one microbe comprising administering an antimicrobial amount of a plurality of peptides and at least one carrier, wherein said peptides are each represented by Formula I: 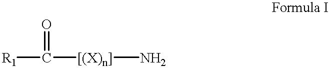
wherein:
X represents any amino acid except glutamate or aspartate;
n=6;
R₁ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₁-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₂ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₁-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₃ is independently hydrogen; C₁-C₄ alkyl; or phenyl optionally substituted with at least one R₈;
R₄ is independently hydrogen; C₁-C₈ alkyl; or phenyl optionally substituted with at least one R₈;
R₅ is independently C₁-C₆ alkyl; C₁-C₆ alkoxy; C₁-C₆ haloalkyl; halogen; C₂-C₈ alkynyl; C₁-C₆ thioalkyl; phenyl or phenoxy each optionally substituted with at least one R₈; cyano; nitro; C₁-C₆ haloalkoxy; C₁-C₆ haloalkythio; C₂-C₆ alkenyl; C₂-C₆ haloalkenyl; acetyl; CO₂CH₃; or N(C₁-C₂ alkyl)₂;
R₆ is independently methyl; ethyl; methoxy; methylthio; halogen; or trifluoromethyl;
R₇ is independently halogen; and
R₈ is independently halogen; C₁-C₄ alkyl; C₁-C₄ alkoxy; C₁-C₄ haloalkyl; nitro; or cyano;
wherein:
the amino acid in the first position, based on numbered amino acids from N-terminus to C-terminus, is selected from the group consisting of arginine, lysine, methionine, serine, threonine and tryptophan;
the amino acid in the second position, based on numbered amino acids from N-terminus to C-terminus, is selected from the group consisting of arginine, histidine, cysteine, threonine, tyrosine, and tryptophan; and
the amino acids in positions three through six, based on numbered amino acids from N-terminus to C-terminus, are any amino acid;
wherein the first two amino acids of said hexapeptides are other than arginine-arginine, tryptophan-tryptophan, tryptophan-cysteine, tryptophan-lysine, arginine-tryptophan, or threonine-arginine.

25. The method of claim 24 wherein said microbe comprises Burkholderia cepacia.

26. A method for preventing, inhibiting, or terminating the growth of at least one microbe comprising administering an antimicrobial amount of a plurality of peptides and at least one carrier, wherein said peptides are each represented by Formula II: 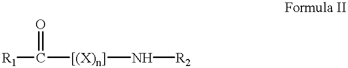
wherein:
X represents any amino acid except glutamate or aspartate;
n=6;
R₁ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₁-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₂ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₁-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₃ is independently hydrogen; C₁-C₄ alkyl; or phenyl optionally substituted with at least one R₈;
R₄ is independently hydrogen; C₁-C₈ alkyl; or phenyl optionally substituted with at least one R₈;
R₅ is independently C₁-C₆ alkyl; C₁-C₆ alkoxy; C₁-C₆ haloalkyl; halogen; C₂-C₈ alkynyl; C₁-C₆ thioalkyl; phenyl or phenoxy each optionally substituted with at least one R₈; cyano; nitro; C₁-C₆ haloalkoxy; C₁-C₆ haloalkythio; C₂-C₆ alkenyl; C₂-C₆ haloalkenyl; acetyl; CO₂CH₃; or N(C₁-C₂ alkyl)₂;
R₆ is independently methyl; ethyl; methoxy; methylthio; halogen; or trifluoromethyl;
R₇ is independently halogen; and
R₈ is independently halogen; C₁-C₄ alkyl; C₁-C₄ alkoxy; C₁-C₄ haloalkyl; nitro; or cyano.

27. The method of claim 26 wherein said microbe comprises Burkholderia cepacia.

28. A composition for coating a substrate comprising an antimicrobial amount of a plurality of hexapeptides and at least one carrier, wherein for each hexapeptide:
the amino acid in the first position, based on numbered amino acids from N-terminus to C-terminus, is selected from the group consisting of arginine, lysine, methionine, serine, threonine and tryptophan;
the amino acid in the second position, based on numbered amino acids from N-terminus to C-terminus, is selected from the group consisting of arginine, histidine, cysteine, threonine, tyrosine, and tryptophan;
the amino acids in positions three through six, based on numbered amino acids from N-terminus to C-terminus, are any amino acid; and
wherein the first two amino acids of said hexapeptides are other than arginine-arginine, tryptophan-tryptophan, tryptophan-cysteine, tryptophan-lysine, arginine-tryptophan, or threonine-arginine.

29. A composition for coating a substrate comprising an antimicrobial amount of a plurality of peptides and at least one carrier, wherein each of said peptides are represented by Formula I: 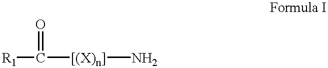
wherein:
X represents any amino acid except glutamate or asparate;
n=6;
R₁ is C₁-C₂₀ alkyl; C₁-C₆ cycloalkyl; C₁-C₂₀ alkenyl; C₁-C₂₀ alkynyl; C₁-C₂₀ haloalkyl; C₁-C₂₀ haloalkenyl; C₁-C₂₀ halo alkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₅-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₂ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₁-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₃ is independently hydrogen; C₁-C₄ alkyl; or phenyl optionally substituted with at least one R₈;
R₄ is independently hydrogen; C₁-C₈ alkyl; or phenyl optionally substituted with at least one R₈;
R₅ is independently C₁-C₆ alkyl; C₁-C₆ alkoxy; C₁-C₆ haloalkyl; halogen; C₂-C₈ alkynyl; C₁-C₆ thioalkyl; phenyl or phenoxy each optionally substituted with at least one R₈; cyano; nitro; C₁-C₆ haloalkoxy; C₁-C₆ haloalkythio; C₂-C₆ alkenyl; C₂-C₆ haloalkenyl; acetyl; CO₂CH₃; or N(C₁-C₂ alkyl)₂;
R₆ is independently methyl; ethyl; methoxy; methylthio; halogen; or trifluoromethyl;
R₇ is independently halogen; and
R₈ is independently halogen; C₁-C₄ alkyl; C₁-C₄ alkoxy; C₁-C₄ haloalkyl; nitro; or cyano;
wherein:
the amino acid in the first position, based on numbered amino acids from N-terminus to C-terminus, is selected from the group consisting of arginine, lysine, methionine, serine, threonine and tryptophan;
the amino acid in the second position, based on numbered amino acids from N-terminus to C-terminus, is selected from the group consisting of arginine, histidine, cysteine, threonine, tyrosine, and tryptophan; and
the amino acids in positions three through six, based on numbered amino acids from N-terminus to C-terminus, are any amino acid;
wherein the first two amino acids of said hexapeptides are other than arginine-arginine, tryptophan-tryptophan, tryptophan-cysteine, tryptophan-lysine, arginine-tryptophan, or threonine-arginine.

30. A composition for coating a substrate comprising an antimicrobial amount of a plurality of peptides and at least one carrier, wherein each of said peptides are represented by Formula II: 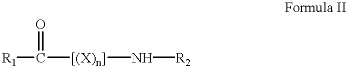
wherein:
X represents any amino acid except glutamate or aspartate;
n=6;
R₁ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₅-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₂ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂ C₂₀ alkylthioalkyl; C₂-C₂₀ alkylsulfinylalkyl; C₂-C₂₀ alkylsulfonylalkyl; C₁-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₃ is independently hydrogen; C₁-C₄ alkyl; or phenyl optionally substituted with at least one R₈;
R₄ is independently hydrogen; C₁-C₈ alkyl; or phenyl optionally substituted with at least one R₈;
R₅ is independently C₁-C₆ alkyl; C₁-C₆ alkoxy; C₁-C₆ haloalkyl; halogen; C₂-C₈ alkynyl; C₁-C₆ thioalkyl; phenyl or phenoxy each optionally substituted with at least one R₈; cyano; nitro; C₁-C₆ haloalkoxy; C₁-C₆ haloalkythio; C₂-C₆ alkenyl; C₂-C₆ haloalkenyl; acetyl; CO₂CH₃; or N(C₁-C₂ alkyl)₂;
R₆ is independently methyl; ethyl; methoxy; methylthio; halogen; or trifluoromethyl;
R₇ is independently halogen; and
R₈ is independently halogen; C₁-C₄ alkyl; C₁-C₄ alkoxy; C₁-C₄ haloalkyl; nitro; or cyano.

31. An antimicrobial composition comprising a plurality of peptides, wherein said peptides each are represented by Formula I: 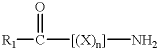
Formula I
wherein:
X represents any amino acid except glutamate or aspartate;
n=1-10;
R₁ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₁-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₃ is independently hydrogen; C₁-C₄ alkyl; or phenyl optionally substituted with at least one R₈;
R₄is independently hydrogen; C₁-C₈ alkyl; or phenyl optionally substituted with at least one R₈;
R₅ is independently C₁-C₆ alkyl; C₁-C₆ alkoxy; C₁-C₆ haloalkyl; halogen; C₂-C₈ alkynyl; C₁-C₆ thioalkyl; phenyl or phenoxy each optionally substituted with at least one R₈; cyano; nitro; C₁-C₆ haloalkoxy; C₁-C₆ haloalkythio; C₂-C₆ alkenyl; C₂-C₆ haloalkenyl; acetyl; CO₂CH₃; or N(C₁-C₂ alkyl)₂;
R₆ is independently methyl; ethyl; methoxy; methylthio; halogen; or trifluoromethyl;
R₇is independently halogen; and
R₈ is independently halogen; C₁-C₄ alkyl; C₁-C₄ alkoxy; C₁-C₄ haloalkyl; nitro; or cyano;
wherein:
the amino acid in the first position, based on numbered amino acids from N-terminus to C-terminus, is selected from the group consisting of arginine, lysine, methionine, serine, threonine and tryptophan;
the amino acid in the second position, based on numbered amino acids from N-terminus to C-terminus, is selected from the group consisting of arginine, histidine, cysteine, threonine, tyrosine, and tryptophan; and
the amino acids in positions three through six, based on numbered amino acids from N-terminus to C-terminus, are any amino acid;
wherein the first two amino acids of said hexapeptides are other than arginine arginine, tryptophan-tryptophan, tryptophan-cysteine, tryptophan-lysine, arginine-tryptophan, or threonine-arginine.

32. An antimicrobial composition comprising a plurality of peptides, wherein said peptides each are represented by Formula II: 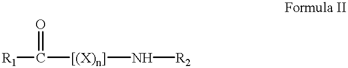
wherein:
X represents any amino acid except glutamate or aspartate;
n=1-10;
R₁ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₁-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₂ is C₁-C₂₀ alkyl; C₃-C₆ cycloalkyl; C₄-C₂₀ alkenyl; C₄-C₂₀ alkynyl; C₁-C₂₀ haloalkyl; C₃-C₂₀ haloalkenyl; C₃-C₂₀ haloalkynyl; C₂-C₂₀ alkoxyalkyl; C₂-C₂₀ alkylthioalkyl; C₂-C₂₀ alkylsulfinylalkyl; C₂-C₂₀ alkylsulfonylalkyl; C₅-C₂₀ cycloalkylalkyl; C₄-C₂₀ alkenyloxyalkyl; C₄-C₂₀ alkynyloxyalkyl; C₄-C₂₀ (cycloalkyl) oxyalkyl; C₄-C₂₀ alkenylthioalkyl; C₄-C₂₀ alkynylthioalkyl; C₆-C₂₀ (cycloalkyl) thioalkyl; C₂-C₂₀ haloalkoxyalkyl; C₄-C₂₀ haloalkenyloxyalkyl; C₄-C₂₀ haloalkynyloxyalkyl; C₄-C₂₀ alkoxylalkenyl; C₄-C₂₀ alkoxyalkynyl; C₄-C₂₀ alkylthioalkenyl; C₄-C₂₀ alkylthioalkynyl; C₄-C₂₀ trialkylsilylalkyl; C₁-C₂₀ alkyl substituted with NR₃R₄, nitro, cyano, or phenyl optionally substituted with R₅, R₆, and R₇; C₁-C₂₀ alkoxy; C₁-C₂₀ haloalkoxy; C₁-C₂₀ alkylthio; C₁-C₂₀ haloalkylthio; NR₃R₄; or phenyl, benzyl, pyridyl, furanyl, thienyl, naphthyl, pyrimidinyl, benzofuranyl, benzothienyl, or quinolinyl each optionally substituted with R₅, R₆ or R₇;
R₃ is independently hydrogen; C₁-C₄ alkyl; or phenyl optionally substituted with at least one R₈;
R₄ is independently hydrogen; C₁-C₈ alkyl; or phenyl optionally substituted with at least one R₈;
R₅is independently C₁-C₆ alkyl; C₁-C₆ alkoxy; C₁-C₆ haloalkyl; halogen; C₂-C₈ alkynyl; C₁-C₆ thioalkyl; phenyl or phenoxy each optionally substituted with at least one R₈; cyano; nitro; C₁-C₆ haloalkoxy; C₁-C₆ haloalkythio; C₂-C₆ alkenyl; C₂-C₆ haloalkenyl; acetyl; CO₂CH₃; or N(C₁-C₂ alkyl)₂;
R₆ is independently methyl; ethyl; methoxy; methylthio; halogen; or trifluoromethyl;
R₇ is independently halogen; and
R₈ is independently halogen; C₁-C₄ alkyl; C₁-C₄ alkoxy; C₁-C₄ haloalkyl; nitro; or cyano;
wherein:
the amino acid in the first position, based on numbered amino acids from N-terminus to C-terminus, is selected from the group consisting of arginine, lysine, methionine, serine, threonine and tryptophan;
the amino acid in the second position, based on numbered amino acids from N-terminus to C-terminus, is selected from the group consisting of arginine, histidine, cysteine, threonine, tyrosine, and tryptophan; and
the amino acids in positions three through six, based on numbered amino acids from N-terminus to C-terminus, are any amino acid.

33. The antimicrobial composition of claim 31 further comprising a carrier selected from the group consisting of a pharmaceutically acceptable carrier, an industrially acceptable carrier, a household product, and a personal care composition.

34. The antimicrobial composition of claim 32 further comprising a carrier selected from the group consisting of a pharmaceutically acceptable carrier, an industrially acceptable carrier, a household product, and a personal care composition.
